(19) United States
(12) Patent Application Publication
(10) Pub. No.: US 2024/0376485 A1
(43) Pub. Date: Nov. 14, 2024

(54) MERISTEM TRANSFORMATION METHOD USING A LIQUID SELECTION MEDIUM

(71) Applicant: Wisconsin Alumni Research Foundation, Madison, WI (US)

(72) Inventors: Edward Williams, Madison, WI (US); William Petersen, Middleton, WI (US); Alvar Carlson, Middleton, WI (US); Ray Collier, Fitchburg, WI (US); Shawn Michael Kaeppler, Oregon, WI (US); Michael W. Petersen, Merrimac, WI (US); Robert Harnish, Middleton, WI (US); Taylor Suo, Middleton, WI (US)

(21) Appl. No.: 18/660,692

(22) Filed: May 10, 2024

Related U.S. Application Data
(60) Provisional application No. 63/501,510, filed on May 11, 2023.

(51) Int. Cl. C12N 15/82

(52) U.S. Cl. CPC C12N 15/8205

(57) ABSTRACT

The present invention provides efficient methods for transforming plant explants with an exogenous nucleic acid by culturing the explant with the exogenous nucleic acid on a liquid selection medium to select for a transformed explant. Transformed Cannabis or other plant explants are also provided. Plants and seeds produced by the methods are also provided and includes production of T1 plants.

### CROSS-REFERENCE TO RELATED APPLICATIONS

This application claims the benefit of and priority to U.S. Provisional Application No. 63/501,510 filed on May 11, 2023, the contents of which is incorporated by reference in its entirety.

### BACKGROUND

Cannabis has been used for millennia for medical and recreational purposes and to create paper, clothing, biofuel, and food. In recent years, the Cannabis industry has grown dramatically in response to expanding legalization and a flood of investor capital. Effective methods for genetically manipulating Cannabis are in high demand, as they would allow desirable traits (e.g., improved disease resistance, increased/decreased production of specific cannabinoids) to be introduced into these plants. However, the use of such methods in Cannabis has been restricted by low rates of transgenic plant regeneration. Thus, more efficient methods for introducing genes into Cannabis are needed in the art.

### SUMMARY

In a first aspect, the present invention provides methods of transforming an explant selected from the group consisting of Cannabis including Cannabis indica, Cannabis sativa, Abelmoschus including Abelmoschus esculentus, L., Gossypium including Gossypium hirsutum, L., Vigna including Vigna unguiculata, L., and Arachis including Arachis hypogaea, L. Common names for the plants include but are not limited to hemp, marijuana, okra, cotton, cowpea and peanut.

The methods comprise (a) excising the explant from a seed by removing the seed coat and optionally cotyledons, (b) introducing the exogenous nucleic acid into the explant, and (c) culturing the explant on a liquid selection medium to select for a transformed explant.

In a second aspect, the present invention provides transformed Cannabis explants produced by the methods described herein.

In a third aspect, the present invention provides Cannabis plants grown from the explants produced by the methods described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Cannabis explants in which both primary leaves were retained during excision. The top panel shows explants that are likely transgene negative (based on their bleaching phenotype) and the bottom panel shows explants that are likely transgene positive (based on their greening phenotype). All explants were grown on 50 ppm liquid spectinomycin.

FIG. 2A-2B show Cannabis explants cultured on a solid hemp node medium. FIG. 2A shows callusing with both embedding (top row) and surface plating (bottom row) in a variant hemp node medium in which the MS salts are replaced with 3.21 g/L Gamborg B5 salts. The gelling agent utilized (i.e., either agar or phytagel) and meta-topolin concentration utilized are indicated above each panel. FIG. 2B shows callusing with both embedding and surface plating (indicated below each panel) in MS-based hemp node media comprising agar (i.e., the medium described in Table 7 plus 8 g/L agar) at the indicated time points. These figures demonstrate that Cannabis explants callus more rapidly when they are embedded in solid hemp node medium as compared to when they are surface plated on it, which suggests that the explants have some sensitivity to this medium.

FIG. 3A-3B show Cannabis explants cultured on liquid medium. FIG. 3A shows the greening phenotype (top photographs) and expression of the fluorescent protein tandem Tomato (tdTomato; tdTOM) (bottom photographs) in an explant cultured on liquid medium for 2 weeks (right side) as compared to an explant grown on solid medium (left side). FIG. 3B shows selection on solid medium (top row) or liquid medium (middle row) after 3 weeks and GUS expression in leaves produced using liquid medium (bottom row).

FIG. 4 shows chimeric phenotypes in some TO Cannabis plants (WP1508-4a,5a) produced using a preliminary version of the Cannabis transformation protocol described herein that utilizes sub-optimal selection.

FIG. 5 shows transformed TO Cannabis plants produced using the Cannabis transformation protocol described herein.

FIG. 6 shows PCR results confirming that the roots of several TO Cannabis plants (i.e., WP1612-5a, WP1331-12a, WP1853-2a, WP1853-3a, and WP1853-5a) produced using the Cannabis transformation protocol described here are transgene positive. WP1507-6a (734) rooted off selection; roots aadA negative by PCR; putative epidermal; WP1612-5a (862) rooted on selection; roots aadA positive by PCR; putative germline; WP1331-12a (DB22) rooted on selection; DNA did not amplify but roots GUS+; putative germline; WP1853-2a,3a (RUBYv1) rooted on selection; roots aadA positive by PCR; both putative germline; WP1853-5a (RUBYv1) rooted on selection; DNA did not amplify but roots RUBYv1 positive; putative germline.

FIG. 7 shows GUS expression in the roots of the Cannabis T0 plant WP1331-12a and RUBYv1 expression in the roots of the Cannabis T0 plant WP1853-5a.

FIG. 8 shows tdTOM expression in roots of the Cannabis T0 plant WP1331-13a. The Cannabis T0 plant WP1612-6a, which comprises a construct that does not include tdTOM, was used to blank the LEICA instrument.

FIG. 9 shows Cannabis explant excision. The top panel shows the removal of the seed coat. The middle panel shows removal of the cotyledons and one or both primary leaves (i.e., the old excision methods). The bottom panel shows removal of only the cotyledons (i.e., the new excision methods).

FIG. 10 shows T1 Germline greenhouse-grown Cannabis seedlings expressing the WCIC-A-862 construct, as observed by greening/bleaching after spraying with 1000 mg/L spectinomycin.

FIG. 11 shows T1 Germline Cannabis seedlings expressing the DICOTBINARY22 (DB22) control construct, as observed by tdTOM presence in the T1 embryo.

FIG. 12 shows T1 Germline Cannabis seedlings expressing the WCIC-A-989 RUBYv1 control construct, as determined by observing betanin presence in the developing plant, or by looking for spectinomycin resistance in developing seedlings.

FIG. 13 shows the results of alternate media schedules, including treatments of feeding Cannabis explants a lower volume of liquid media at greater frequency than our standard treatment.

FIG. 14 shows T0 plants recovered from this GAANTRY (Gene Assembly in Agrobacterium by Nucleic acid sTacking using Recombinase technologY) system, where experiments in Cannabis meristems employed T-DNA launched from the disarmed virulence/Ri plasmid rather than T-DNA launched from a binary plasmid.

FIG. 15 shows the first Cannabis T0 plants from GAANTRY rooted in the presence of spectinomycin, with one plant expressing tdTOM and the other expressing GUS.

FIG. 16 shows Cannabis transformation metrics from conventional binary strategy compared to GAANTRY.

FIG. 17 shows transient GUS expression in TO okra meristem explants post co-culture (right panel), compared to a non-inoculated control (left panel) using the Efficient Cannabis Transformation process.

FIG. 18 shows Okra explant phenotypes on non-selective MS liquid media (far left image of left panel) and on a solid B5 media (right panel). Variable spectinomycin concentrations (0, 25 or 50 mg/L) are indicated above the photographs.

FIG. 19 shows stable tdTOM expression in TO Okra roots of the first plant 7.5 weeks post-inoculation. Explants followed the “Efficient Cannabis meristem tfn protocol”.

FIG. 20 shows TO Okra phenotypes from plants generated with the “Efficient Cannabis transformation” process.

FIG. 21 shows Stable tdTOM (roots) and GUS expression (roots, leaves) in TO Okra plants derived from “Efficient Cannabis transformation” process. Images captured 8.5 weeks post-inoculation. Explants followed “Efficient Cannabis meristem tfn protocol” with 25 mg/L active spectinomycin during regeneration/selection.

FIG. 22 shows stable tdTOM expression in TO Okra event WP2300-3a (right plant in both panels) ˜1 month after handoff; the control plant is the left plant in both panels.

FIG. 23 shows examples of T0 Okra plant phenotypes in the greenhouse.

FIG. 24 shows Okra conventional pod/seed (left panel) vs. tdTOM expression in T1 Okra pod/seed of WP2300-4a (middle and right panels).

FIG. 25 shows Okra conventional seeds (top left panel) and conventional split seeds (top right panel) vs. tdTOM expression in T1 Okra seeds (bottom left panel) and tdTOM expression in T1 Okra split seeds (bottom right panel) of WP2300-4a.

FIG. 26 shows okra meristem explants on 25 ppm spec (left); 50 ppm spec (right) on solid (top) or liquid (bottom) Hemp node media (˜3 weeks post inoculation).

FIG. 27 shows phenotypes of Cotton meristem explants on non-selective solid B5 (right), and on liquid B5 (left) after ˜2 weeks.

FIG. 28 shows TO Cowpea seedlings expressing DICOTBINARY22 (DB22) and DICOTBINARY52 (DB52) using hydroponic/liquid selection media regime analogous to Efficient Cannabis meristem method (right) compared to standard semisolid selection media regime (left).

FIG. 29 Cowpea variety “Crowder Pea” events with a brief liquid delay phase followed by liquid selection with spectinomycin. Treatments include transferring explants to solid selection media after co-culture (std), and explants transferred to liquid media without selection for 3 days (delay), followed by transfer to liquid media with 5-25 mg/L spectinomycin selection.

FIG. 30 shows stable tdTom expression in leaves of Cowpea (Crowder pea) events generated on solid selection media (std) and from liquid media using a delay phase (3d delay followed by 5-25 mg/L spectinomycin selection).

FIG. 31 shows presence/absence of GUS expression in the vascular bundles of cross-sectioned cowpea petioles to predict germline status.

FIG. 32 shows peanut meristem explants.

FIG. 33 shows peanut meristem explants on solid (top) and liquid (middle and bottom) MS-based Cannabis node selection medias post co-culture with 0 mg/L (left), 25 mg/L (middle) and 50 mg/L (right) of spectinomycin approximately 2.5 weeks after inoculation.

FIG. 34 shows stable GUS expression in highly chimeric peanut shoots sonicated for 10 minutes (45 kHz) in the presence of Agrobacterium rhizogenes strain 18r12v (Ar18r12v)/DB22 inoculum and vacuum infiltrated. 4 day co-culture in 2.5 mL INO+lipoic acid+nystatin/TBZ+1 mg/L TDZ; 23° C. 16/8 photoperiod. Selection/regeneration on liquid Hemp Node media with 50 mg/L spectinomycin varying amounts of meta-topolin (0, 0.5, 1, or 2 mg/L meta-topolin). SAM removed after 1 week on selection; transferred to WPM after 1 month of liquid selection.

### DETAILED DESCRIPTION

The present invention provides efficient methods for transforming an explant selected from the group consisting of Cannabis sativa, (hemp), Abelmoschus esculentus, L. (okra), Gossypium hirsutum, L. (cotton), Vigna unguiculata, L. (cowpea), and Arachis hypogaea, L. (peanut). While the examples provided herein demonstrate the methods described in Cannabis, okra, cotton, cowpea and peanut, those of skill in the art will appreciate that the methods provided herein may be used with other plants from similar plant species or plants from the following genera: Cannabis, Abelmoschus, Gossypium, Vigna, and Arachis. Transformed explants and plants produced by the methods are also provided.

In a previous patent application, which was granted as U.S. Pat. No. 11,512,320, the present inventors describe a method for transforming Cannabis meristem explants. This method produces confirmed germline events, but it requires prolonged tissue culture and laborious explant transfers, and it generally produces transformation frequencies of less than 1%. These low transformations frequencies can be at least partially attributed to poor rooting and the fact that many explants would develop a necrotic growing tip.

In the present application, the inventors describe an improved method for transforming Cannabis and the application of this new method to other plants. The new method offers several key benefits as compared to the old method: (1) it produces greenhouse-ready plants in significantly less time, (2) it results in a 5- to 10-fold higher transformation frequency, and (3) it requires far less manual manipulation of explants (i.e., during both explant excision and culturing). As a result, the new method is more amenable to automation and requires fewer highly skilled personnel hours per transformed plant. In some embodiments, the method may be used to produce greenhouse-ready plants in less than 5 months, less than 4 months, less than 120 days, less than 110 days, less than 100 days. A detailed comparison of the old and new transformation methods is provided in the Examples.

The inventors also applied the method for transforming Cannabis to additional plant species, including an explant selected from the group consisting of Cannabis sativa, Abelmoschus esculentus, L., Gossypium hirsutum, L., Vigna unguiculata, L., and Arachis hypogaea, L. Modifications to the methods to optimize transformation efficiencies for individual species are provided herein.

### Methods:

In a first aspect, the present invention provides methods of transforming an explant. As used herein, the term “transformation” refers to the genetic alteration of a cell via the direct uptake and incorporation of an exogenous nucleic acid. The methods of the present invention comprise (a) excising the explant from a seed by removing the seed coat and optionally cotyledons, (b) introducing the exogenous nucleic acid into the explant, and (c) culturing the explant on a liquid selection medium to select for a transformed explant.

Cannabis, which is also known as hemp, is a genus of flowering plants in the family Cannabaceae. The methods of the present invention utilize a Cannabis seed. A “seed” is an embryonic plant enclosed in a protective outer covering. The seed used in the present methods may be from any Cannabis cultivar of interest. For example, the seed may be from Cannabis sativa, Cannabis indica, or a variety developed by crossbreeding Cannabis sativa and Cannabis indica. The seed used in the present methods may also be from any cultivar of Abelmoschus esculentus, L. (okra), Gossypium hirsutum, L. (cotton), Vigna unguiculata, L. (cowpea), or Arachis hypogaea, L. (peanut).

In some embodiments, the methods may further comprise sanitizing the seed prior to step (a). Any sanitization method known in the art may be used. As used herein, “sanitization” refers to a process that removes, kills, or deactivates microorganisms. Sanitization can be achieved through various means, including heat, radiation, ultraviolet (UV) light, oxidizing gasses, plasma, high pressure, and disinfection agents. Suitable disinfection agents include, but are not limited to, chlorine, sodium hypochlorite, alcohol, and hydrogen peroxide. In the Examples, the inventors sanitized seed by incubating it in 20% Clorox™ bleach for 5 minutes. Thus, in some embodiments, the seed is sanitized using bleach (i.e., sodium hypochlorite). However, the inventors have also successfully sanitized seeds by heating them in a 50° C. water bath for 20 minutes. Thus, in some embodiments, the seed is sanitized using heat. Additional embodiments include sanitizing the seed with sulfuric acid or 15d at 4 degrees Celsius, or a combination of sulfuric acid and cold treatment, described by Liberatore et al. (2018). Thus, in some embodiments, the seed is sanitized using sulfuric acid and/or cold treatment.

In some embodiments, the methods may further comprise hydrating the seed in a hydration medium prior to step (a). The term “hydration” refers to a process in which a dry seed takes up (i.e., imbibes) water. As a seed imbibes water, enzymes within the seed are activated, increasing the metabolic activity of the seed, and preparing the seed for germination. In some embodiments, the seed is hydrated for a time sufficient for the seed to reach a moisture content of between 30% and 70%. In some embodiments, the seed is hydrated for at least 12 hours. In some embodiments, the seed is hydrated between 2 and 24 hours. The hydration step may be completed after the sanitization step.

The “hydration medium” used to hydrate the seed may be any sterile medium that supports survival of the meristematic tissue in the seed. For example, the hydration medium may comprise sterile water and/or a sterile tissue culture medium. In the Examples, the inventors utilized a hydration medium comprising sterile water, cefotaxime (antibacterial agent), Captan® (antifungal agent), and Bravo® (antifungal agent). Thus, in some embodiments, the hydration media comprises antibacterial agents (i.e., agents that kill bacteria or inhibit bacterial growth and/or reproduction) and/or antifungal agents (i.e., agents that kill fungi or inhibit fungal growth and/or reproduction).

In some embodiments, the hydration medium comprises one or more growth regulators. A “growth regulator” is a chemical that can be used to modify plant growth. For instance, growth regulators can be used to increase branching, increase rooting, suppress shoot growth, increase yields, and the like. Examples of growth regulators that can be used in the methods of the present invention include, but are not limited to, thidiazuron (TDZ), 6-benzylaminopurine (BAP), polyethylene glycol (PEG), 2,4-dichlorophenoxyacetic acid (2,4-D), PACZOL®, gibberellic acid (GA3), indole-3-acetic acid (IAA), indole-3-butyric acid (IBA), 1-naphthalaneacetic acid (NAA), forchlorfenuron (CPPU), glyphosate, glufosinate, bialophos, hygromycin, amikacin, tobramycin, imazapyr, dicamba, polyvinylpyrrolidone (PVP), polyvinylpolypyrrolidone (PVPP), salicylic acid, proline, betaine, ethylene, brassinosteroids, nitrates, meta-topolin (mT), and gibberellins.

In the Examples, the inventors sanitized seeds and then hydrated them in a hydration medium before excising explants from them. Thus, in some embodiments, the method comprises sanitizing the seed and then hydrating the seed in a hydration medium prior to step (a). The inventors contemplate using a physical means to remove the seed coat would also be suitable instead of the hydration step. In either case the sanitization step is optional and can be completed in various ways as described above.

In step (a) of the present methods, an explant is excised from the seed. As used herein, the term “explant” refers to a cell or tissue that is removed from a seed and used to initiate a culture in vitro. Explants comprise meristematic tissue, which consists of undifferentiated cells that can give rise to all adult plant tissues. Plant tissues that can be used as explants include, without limitation, embryos, cotyledons, hypocotyls, leaf bases, mesocotyls, plumules, protoplasts, and embryonic axes. Explant excision may be accomplished, for example, via manual processing (e.g., using knives and forceps), wet milling using a series of rollers and spray nozzles, adjustable grinding plates, pressure, injected gasses, vacuum, or turbulence.

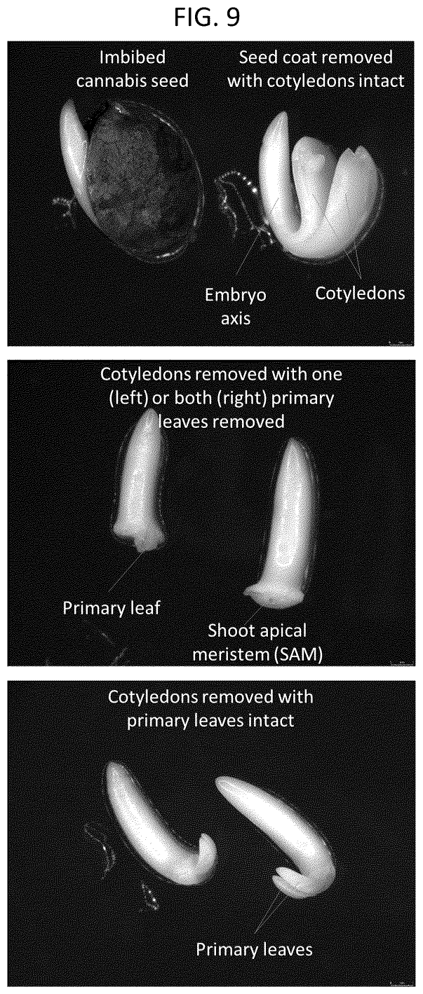

In preferred embodiments with respect to Cannabis, the explant comprises both primary leaves. In their previous Cannabis explant excision protocol, the inventors manually removed the seed coat, cotyledons, and one or both primary leaves from a seed to form an explant (see FIG. 9, middle panel). However, as is described in the Examples, the inventors discovered that they could streamline this protocol (i.e., reduce the amount of manual labor required) by retaining the primary leaves. Thus, the inventors' new explant excision protocol comprises removing only the seed coat and cotyledons from the seed (see FIG. 9, bottom panel). Modifications of the Cannabis explant excision protocol were tested for explants of the additional plant species provided in the Examples. Variations included, in some cases, removal of none, only one or both primary leaves. In some embodiments leaving the cotyledon intact was beneficial. The results of these experiments are presented in the Examples.

In step (b) of the methods, an exogenous nucleic acid is introduced into the explant. As used herein, “introducing” describes a process by which exogenous nucleic acids are introduced into a recipient cell. Suitable introduction methods include, without limitation, bacteria-mediated transformation, transposition-based plant transformation, the floral dip method, viral infection (e.g., using tomato yellow leaf curl virus, tobacco yellow dwarf virus, tomato golden mosaic virus, or bean pod mottle virus), electroporation, heat shock, lipofection, microinjection, high velocity microprojection, vacuum-infiltration, direct DNA uptake, and particle bombardment. Bacteria that can be used for bacterial-mediated transformation include several species of Rhizobiaceae such as Agrobacterium spp., Sinorhizobium spp., Mesorhizobium spp., Rhizobium spp., Ochrobacterium spp., and Bradyrhizobium spp. In the Examples, the inventors transformed Cannabis explants using Agrobacterium rhizogenes strain 18r12v (Ar18r12v). Thus, in some embodiments, the exogenous nucleic acid is introduced via Agrobacterium-mediated transformation.

In Agrobacterium-mediated transformation the Transfer DNA (T-DNA), an exogenous nucleic acid is delivered into plant cells as part of a binary Agrobacterium vector in which it is flanked by two imperfect border repeat sequences (the Right and Left Borders; RB and LB, respectively). Prior to transformation into plant cells, this binary vector is co-transformed into Agrobacterium with a second vector, which must have an origin of replication which is from a different incompatibility group than that used for replication of the binary plasmid, referred to as a vir helper plasmid. The vir helper plasmid encodes proteins that mediate integration of the nucleic acid flanked by the T-DNA repeats into the genome of the plant cell. Thus, to introduce an exogenous nucleic acid into an explant via Agrobacterium-mediated transformation, the explant is co-cultured in a co-culture medium with an Agrobacterium comprising a vector comprising the exogenous nucleic acid for about 1 to 6 days. In some embodiments, the explant is co-cultured with the Agrobacterium for about 4 days.

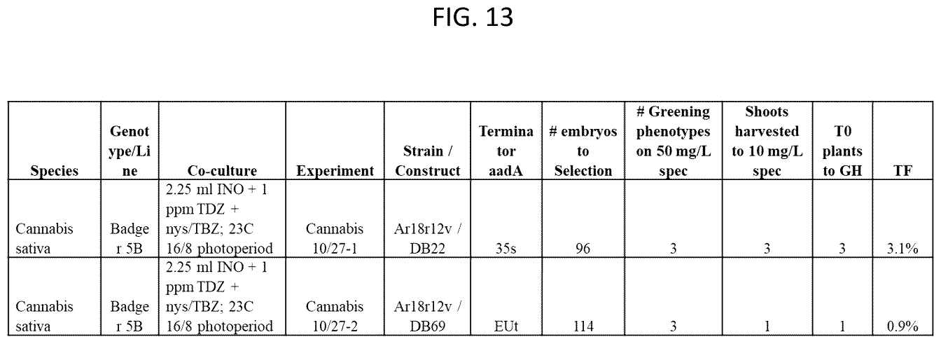

In the Examples, Cannabis explants were transformed with an exogenous nucleic acid comprising the aadA gene. In some embodiments, an alternate terminator was used for the aadA cassette. Based on work by Diamos and Mason, (Diamos and Mason, 2018) we also examined using an alternate terminator on the aadA cassette (the EUt terminator against the standard 35s terminator on DICOTBINARY22). Although we did not see an advantage with the EUt terminator, we did obtain a T0 plant from its use and it offers an alternate embodiment to our selection cassette (FIG. 13).

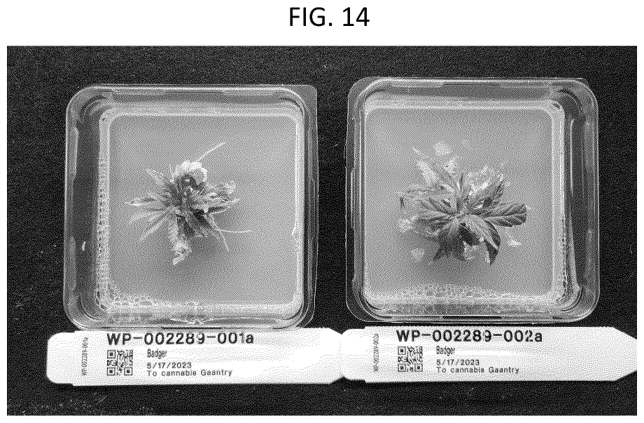

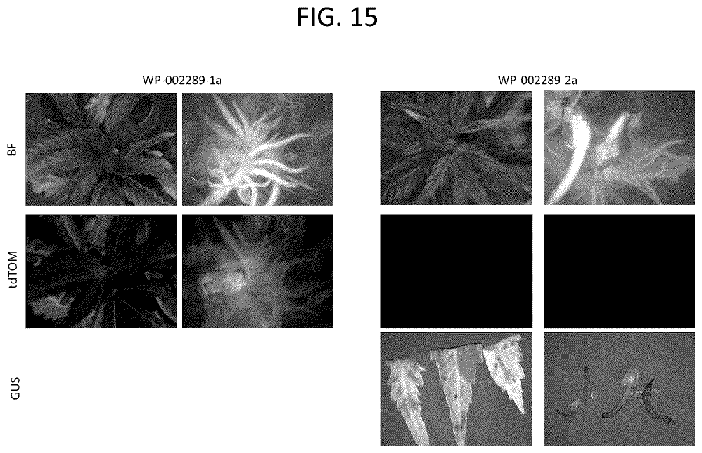

In some embodiments, the GAANTRY (Gene Assembly in Agrobacterium by Nucleic acid sTacking using Recombinase technologY) system may also be used for transformation of explants. We ran proof of concept experiments in Cannabis meristems using T-DNA launched from the disarmed virulence/Ri plasmid (Collier 2018) rather than T-DNA launched from a binary plasmid and were able to recover T0 plants from this GAANTRY (Gene Assembly in Agrobacterium by Nucleic acid sTacking using Recombinase technologY) system (FIG. 14). The first TO plants from GAANTRY rooted in the presence of spectinomycin, with one plant expressing tdTOM and the other expressing GUS, are shown in FIG. 15.

The “co-culture medium” used for Agrobacterium-mediated transformation may be any medium that supports the growth and survival of the explant. In some embodiments, the co-culture medium comprises one or more growth regulators (see examples of growth regulators above). In the Cannabis Examples, the inventors utilized the co-culture medium described in Table 6, which comprises dicot INO medium, nystatin (antifungal agent), thiabendazole (antifungal agent), and thidiazuron (growth regulator). Thus, in some embodiments, the co-culture medium comprises the growth regulator thidiazuron. The co-culture medium may be modified, or alternative co-culture mediums may be used for different tissues or species. As described in the Examples below, we retained the 1 mg/L TDZ in INO-based co-culture, but other cytokinins (ex. BAP) could be used in co-culture and at different concentrations. In addition, solidified co-culture media could be utilized by adding a solidifying agent, such as agar, agarose, phytagel or others to INO media.

In some embodiments, the methods further comprise force treating the explant prior to or following step (b) to aid in the uptake of the exogenous nucleic acid. Examples of suitable force treatment methods include, without limitation, sonication, vortexing, centrifugation, heat-shock, increased pressure, vacuum infiltration, desiccation, and addition of chemicals (e.g., TDZ, glyphosate, metolachlor). In the Examples, the inventors force treated explants via sonication at 45-55 kHz for 20 seconds. Thus, in some embodiments, the explants are sonicated.

In step (c) of the methods, the explant is cultured on a liquid selection medium to select for transformed explants. A “selection medium” is a medium that comprises a selection agent. A “selection agent” is an agent that changes the phenotype, kills, or prevents the growth of cells that do not comprise a selectable marker (i.e., a gene that protects cells from an otherwise toxic compound). Thus, ideally, only explants that are transformed with a selectable marker can grow on the selection medium. Examples of suitable selection agents include antibiotics (e.g., spectinomycin, streptomycin) and herbicides (e.g., imazapyr). In the Examples, the explants were transformed with an exogenous nucleic acid comprising the aadA gene, which confers resistance to spectinomycin, and spectinomycin was used in the selection medium. Thus, in preferred embodiments, the selection medium comprises spectinomycin. While 50 mg/L of spectinomycin was used in the liquid selection medium in the Examples, the inventors have also achieved bleaching of non-transformed cells with as little as 10-15 mg/L spectinomycin and have used up to 150 mg/L spectinomycin in other dicot meristem systems. Thus, in some embodiments, the selection medium comprises 10-150 mg/L spectinomycin. In other embodiments, the selection medium comprises 20-100 mg/L, 30-80 mg/L, or 40-60 mg/L spectinomycin.

Any liquid medium that supports the growth and survival of transformed explants may be used as the selection medium. Suitable base media for use in the selection medium include, without limitation, B5 medium, DKW, WPM-based medium, MS salts-based medium, and ½×MS salts-based medium. Different plants and tissues may require different base media selected from the group consisting of B5 medium, DKW medium, WPM-based medium, MS salts-based medium, and ½×MS salts-based medium, and possibly further modifications necessary, as described below and in the Examples. In addition to the base medium, the selection medium should comprise at least one selection agent and may additionally comprise additives such as antibacterial agents, antifungal agents, growth regulators, and micronutrients. In the Examples, the inventors used the selection medium described in Table 7, which includes MS salts, sucrose, Cleary's 3336 (antifungal agent), meta-topolin (growth regulator), carbenicillin (antibacterial agent), cefotaxime (antibacterial agent), timentin (antibacterial agent), and spectinomycin (selection agent). Additional embodiments of the selection medium may contain ammonium nitrate and potassium nitrate, or both. In some embodiments, the liquid selection medium is hemp node media (MS-based) and comprises 1600-3000 mg/L ammonium nitrate. In a preferred embodiment, the hemp node medium comprises 2500 mg/L ammonium nitrate. In some embodiments, the liquid selection medium is DKW and comprises 0-1500 mg/L potassium nitrate. In a preferred embodiment, the DKW medium comprises 950 mg/L potassium nitrate.

The selection medium used with the present invention is a liquid selection medium, meaning that it does not solidify at room temperature. Thus, the selection medium used with the present invention may not comprise agar or other gelling agents. Cannabis explants may form callus when cultured on the agar-based hemp node medium that was used as the selection medium in the inventors' previous Cannabis transformation method (i.e., the method described in U.S. Pat. No. 11,512,320). This previous method was labor intensive, as it required that the that the explants were transferred one-by-one to fresh solid media every 2-3 weeks. In addition, it also required callus to be manually removed with a scalpel in some cases also greatly increasing the workload. As is described in Example 1, the inventors discovered that using a liquid formulation of hemp node medium as the selection medium minimized the time required to provide fresh media and also reduces callusing to the extent that callus removal is unnecessary. With liquid selection medium, explants can be passaged (i.e., transferred to fresh media) by simply adding fresh media to the culture dish rather than moving each fragile explant to a new culture dish by hand. Spent media may be removed from the culture dish prior to adding fresh media. Thus, the use of liquid selection medium dramatically decreases the amount of manual labor required in this step of the method because the explant are not moved from one culture dish to a fresh culture dish. In some embodiments, the explants are not transferred to a new culture dish during the selection process. This also decreases the cost of supplies for use in the methods as compared to methods in which the explants must be transferred to new culture dishes every 2-3 weeks. In some embodiments, a delay between steps (b) (introducing the nucleic acid) and step (c) (culturing in the liquid selection medium) of the method may be employed. The delay may be 1, 2, 3, 4, or 5 days or longer. In a preferred embodiment, a three-day delay is employed.

In subsequent experiments, the inventors tested alternate media schedules, modified media, and additional plant species. In addition, transformation frequencies for T1 Cannabis plants are provided in Example 3. As described in Example 4, alternate media schedules involving feeding explants a lower volume of liquid media at greater frequency than the standard treatment did not appear advantageous save for offering greater flexibility to the feeding schedule (Table 11). Also described in Example 4, the inventors examined alternate medias during the selection/regeneration phase (Table 12). The first set of these experiments examined varying levels of ammonium nitrate and potassium nitrate in the media. However, lowering the ammonium nitrate concentration did not appear advantageous over the standard (although in this set the standard treatment did not produce TO plants). The inventors did obtain a T0 plant by increasing the ammonium nitrate concentration from the std MS level (1650 mg/L) to 2500 mg/L. Additionally, plants were regenerated using DKW media, which has a comparable level of ammonium nitrate but a lower amount of potassium nitrate than MS media. The inventors also examined the impact of Phytoax cytokinin replacing meta-topolin in the regenerative media. However, Phytoax did not appear advantageous over meta-topolin, but the experiment did demonstrate generation of T0 plants using DKW media as an alternative to MS media. The inventors then tested using one or more liquid selection mediums, including a liquid formulation of hemp node medium, as the selection medium for other plant species, and in most cases found superior results compared to using a solid medium. In some embodiments, modifying the liquid hemp node medium produced better results, depending on the species. In other embodiments, using an alternative liquid selection medium other than the liquid hemp node medium produced better results. Example 5 shows successful germline TO Okra transgenic plant production through the Efficient Cannabis Transformation process, illustrating an advantage from using liquid selection medium. Example 6 shows greater regeneration of Cotton explants when grown on a liquid medium. Example 7 illustrates successful TO cowpea transgenic plant production using a hydroponic/liquid media regime analogous to the Efficient Cannabis Transformation process with modifications, including a 3-day liquid delay phase prior to transferring to the liquid selection medium. Example 8 describes the results of testing Peanut meristem explants on solid and liquid MS-based Cannabis node selection medias post co-culture, with an advantage to using the liquid medium. Although stable TO Peanut plants were not recovered in these experiments, recovery of regenerating highly chimeric Peanut plants stably expressing GUS does suggest feasibility of this strategy to those skilled in the art. These experiments demonstrate that different timing of steps and feeding schedules, different media compositions and different growth regulators may be used and still achieve the improvements in transformation efficiency described herein by using a liquid selection medium in step (c) of the method.

In some embodiments, the methods further comprise (d) culturing the transformed explant on a rooting medium. Any medium that supports the growth and rooting of transformed explants may be used as the rooting medium. Suitable base media for use in the rooting medium include, without limitation, woody plant medium (WPM)-based medium, ½×Murashige and Skoog (MS)-based medium, Linsmaier and Skoog (LS) medium, White's Medium, and Gamborg (B5) medium. Ideally, the rooting medium comprises rooting auxins, such as indole acetic acid (IAA), indole-3-butyric acid (IBA), and naphthalene acetic acid (NAA). In the Examples, the inventors demonstrate that the use of a WPM-based rooting medium enhanced the level and rate of rooting as compared to the ½×MS-based rooting medium used in the previous method. Thus, in some embodiments, the rooting medium is WPM-based. In addition to the base medium, the rooting medium may further comprise additives such as antibacterial agents, antifungal agents, growth regulators, gelling agents, and selection agents. In the Examples, the inventors used the rooting medium described in Table 8, which includes WPM salts, sucrose, agar (gelling agent), IBA (growth regulator), cefotaxime (antibacterial agent), timentin (antibacterial agent), and spectinomycin (selection agent).

In the Examples, the inventors tested the minimal level of the selection agent spectinomycin that could be used in the rooting medium to allow for selection of successful transformants and found that 10 mg/L spectinomycin is sufficient while 5 mg/L spectinomycin allows non-transgenic shoots to root. However, the inventors have successfully produced transgenic Cannabis plants using rooting media containing concentrations of spectinomycin ranging from 0 to 60.2 mg/L. Thus, in some embodiments, the rooting medium comprises 5-100 mg/L, 7-60 mg/L, or 9-11 mg/mL spectinomycin.

The methods of the present invention offer several major advantages over the inventors' previous method for transforming Cannabis (i.e., the method described in U.S. Pat. No. 11,512,320). One such advantage is that the methods of the present invention produce greenhouse-ready plantlets in less than 100 days post-inoculation. In the Examples, the new methods produced greenhouse-ready plantlets within 60-71 days of inoculation, whereas the old methods produced greenhouse-ready plantlets within 103-255 days of inoculation (Table 2). Thus, the new method reduces the time to greenhouse by at least 30 days as compared to the old method. In some embodiments, the methods of the present invention produce greenhouse-ready plantlets in less than 90 days, less than 85 days, less than 80 days, less than 75 days, less than 70 days, less than 65 days, less than 60 days, less than 55 days, or less than 50 days. A plantlet is considered “greenhouse-ready” after it has developed roots that are at least 2 cm long and leaves.

Another advantage is that the methods of the present invention have a transformation frequency of greater than 1%. In Examples 1 and 2, the new methods produced transformation frequencies ranging from 1.5 to 3.8% whereas the old methods produced transformation frequencies ranging from 0.1 to 0.3% (Table 2). Thus, the new methods have a transformation frequency that is about 5- to 10-fold higher than that of the old methods. In some embodiments, the methods have a transformation frequency of 1-5%. Example 3 describes further work producing stable T1 Cannabis plants, while previously, germline rates (T1) were predicted from TO Cannabis shoots rooting on selection and/or presence of transgene in TO root tissue. The production of T1 Cannabis plants having transformation frequencies of 1-5% provides a significant improvement in Cannabis transformation efficiency, especially given the difficulty of transforming this incalcitrant species. Transformation efficiencies for additional species tested herein are also provided in the Examples. “Transformation frequency” is calculated by dividing the number of T0 or T1 plants produced by the number of T0 or T1 explants inoculated, respectively.

In the methods of the present invention, Cannabis explants are transformed with an exogenous nucleic acid. The terms “nucleic acid,” “oligonucleotide,” and “polynucleotide” are used interchangeably to refer a polymer of DNA or RNA. A nucleic acid may be single-stranded or double-stranded and may represent the sense or the antisense strand. A nucleic acid may be synthesized or obtained from a natural source. The nucleic acids used with the present invention are “exogenous,” meaning that they originate outside of Cannabis or would represent inclusion of an additional copy of a Cannabis-derived nucleic acid from the same or a different variety of Cannabis.

The exogenous nucleic acid used with the present invention may include a novel nucleic acid that is not found in the Cannabis genome, a modified version of a nucleic acid found in the Cannabis genome, or an extra copy of a nucleic acid found in the Cannabis genome. In some embodiments, the exogenous nucleic acid is used to reduce or silence the expression of a nucleic acid found in the Cannabis genome, e.g., via RNA interference (RNAi). In some embodiments, the exogenous nucleic acid encodes or includes a guide RNA (gRNA) that is used to perform CRISPR/Cas-mediated gene editing (CRISPR) on the Cannabis genome. CRISPR can be used to edit an endogenous gene (e.g., correct a mutation or modify the product produced by the gene), disrupt expression of an endogenous gene (e.g., by inserting a stop codon, a frameshift mutation, or a nonsense mutation), modify a regulatory sequence to upregulate or downregulate expression of an endogenous gene, or insert an exogenous gene (e.g., a gene encoding a novel product). In these embodiments, the exogenous nucleic acid may further encode a Cas enzyme or a Cas enzyme may be introduced by other means.

The exogenous nucleic acid used with the present invention may confer a desirable trait or phenotype to the transformed Cannabis plant. In some embodiments, the exogenous nucleic acid confers a trait of agronomic interest, such as resistance to a disease, insect, or pest; tolerance to an herbicide or environmental stress; growth enhancement (e.g., increased plant size, growth rate, or nitrogen fixation), or a plant product improvement (e.g., increased yield, nutritional enhancement, improved flavor, altered fruit ripening). In some embodiments, the exogenous nucleic acid causes the plant to produce a novel product (e.g., a pharmaceutical, an industrial enzyme).

In some embodiments, the exogenous nucleic acid modulates the expression or activity of an endogenous Cannabis gene selected from the group consisting of tetrahydrocannabinolic acid (THCA) synthase, cannabidiolic acid (CBDA) synthase, 5-enolpyruvylshikimate-3-phosphate (EPSP) synthase, O-methyltransferase (CsOMT21), lipid transfer protein 2 (LTP2), prenyltransferase 3 (CsPT3), and prenyltransferase 1 (CsPT1). For example, Cannabis plants that have low THC content can be generated by reducing or eliminating expression of THCA synthase and/or CBDA synthase; Cannabis plants with increased trichome numbers can be generated by increasing expression of LTP2; Cannabis plants with increased cannabigerol (CBG) and cannabidiol (CBD) production can be generated by increasing expression of CsPT1 or CsPT3; Cannabis plants with increased chrysoeriol, cannflavin A, and cannflavin B production can be generated by increasing expression of CsOMT21; and glyphosate resistant Cannabis plants can be generated by mutating the 5-enolpyruvylshikimate-3-phosphate (EPSP) synthase (EPSPS) gene. The sequences of these genes and the proteins that they encode as well as examples of gRNAs that can be used to target the THCA and CBDA genes are provided in U.S. Pat. No. 11,512,320, which is hereby incorporated by reference in its entirety.

In some embodiments, the exogenous nucleic acid comprises a promoter or another regulatory element. As used herein, the term “promoter” refers to a DNA sequence that defines where transcription of a nucleic acid begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or exogenous gene, may be composed of elements derived from multiple regulatory sequences found in nature, or may comprise synthetic DNA. A promoter is “operably linked” to a nucleic acid if the promoter is positioned such that it can affect transcription of the nucleic acid.

### Explants and Plants:

In another aspect, the present invention provides transformed explants produced by the methods described herein.

The present invention also provides plants grown from the explants produced by the methods described herein. The term “plant” is used broadly herein to refer to a plant at any stage of development or to part of a plant, including a plant cutting, a plant cell, a plant cell culture, a plant organ, a plant tissue, a plant seed, a plantlet, or a harvestable plant part (e.g., flowers, pollen, seedlings, cuttings, tubers, leaves, stems, fruit, seeds, roots).

In preferred embodiments, the explants or plants produced by the methods are germline transformants. A “germline transformant” is a transformed explant or plant in which the exogenous nucleic acid has been transformed into cells that will give rise to pollen or an ovule, such that the exogenous nucleic acid is passed on to seed produced by the plant.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventors describe an improved method for transforming Cannabis.

### Protocol Modifications:

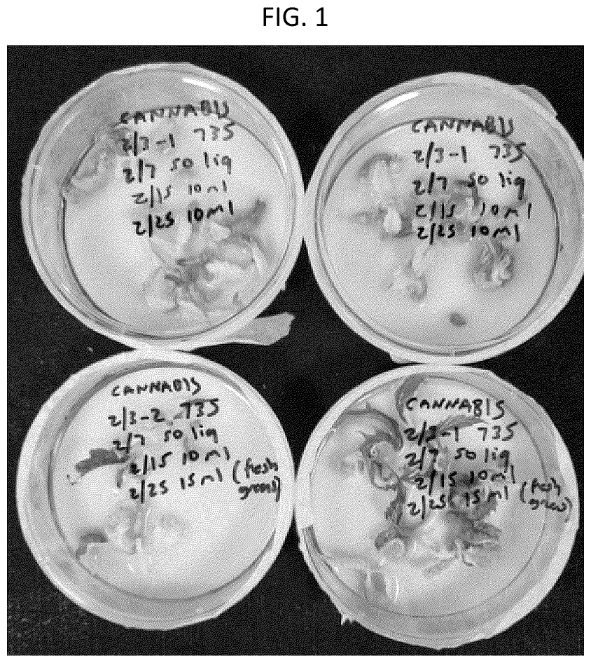

Simplified excision method. Retaining both primary leaves of the Cannabis explant during excision increases the efficiency of isolating this relatively delicate tissue. Both greening phenotypes and T0 Cannabis plants have been obtained using this simplified excision method (FIG. 1).

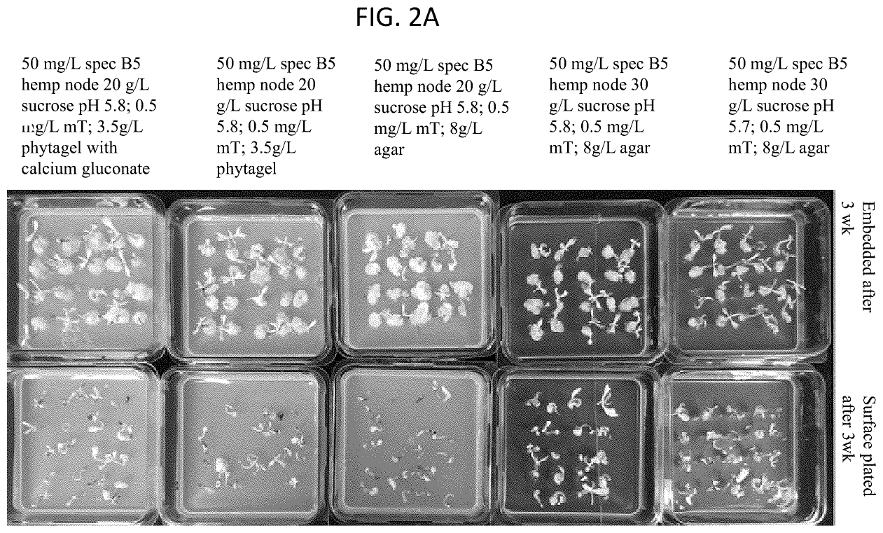

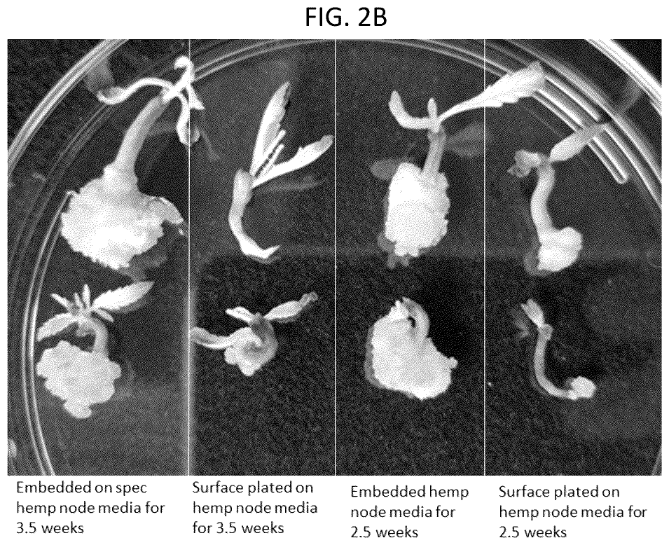

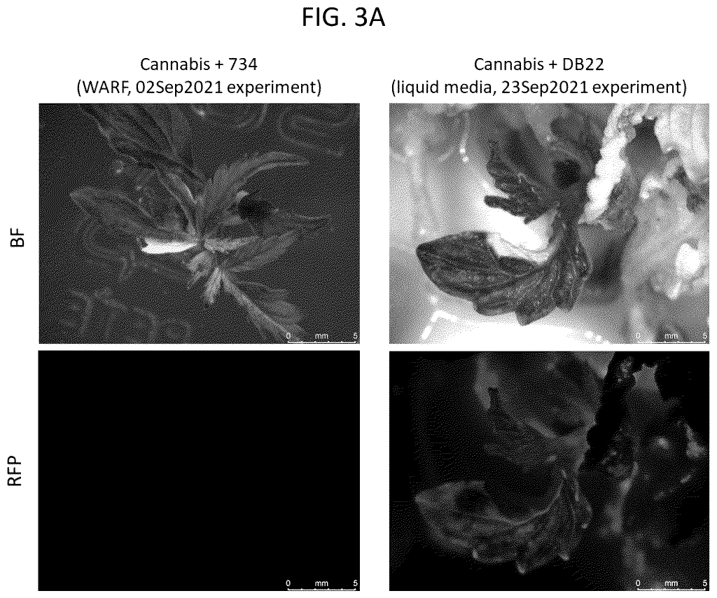

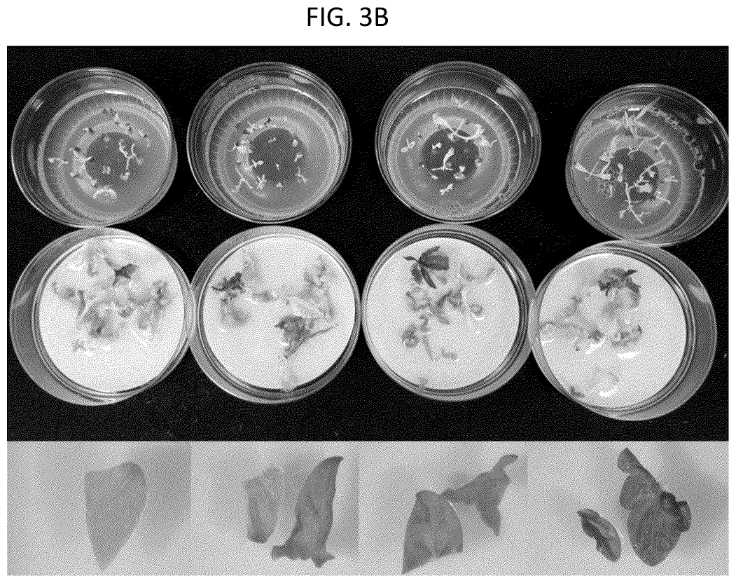

Liquid selection medium. Cannabis explants undergo extensive callusing (i.e., unorganized callusing due to hyperhydricity as opposed to embryogenic or organogenic callus) at the hypocotyl when cultured on agar-based hemp node medium post co-culture, which requires labor-intensive manual callus removal with a scalpel every 2-3 weeks for every explant. Phytagel-based hemp node medium and surface plating were tested as alternatives to agar-based hemp node medium. These modifications did not reduce callusing, but surface plating did delay callusing slightly (FIG. 2). However, a liquid formulation of hemp node medium was found to dramatically reduce callusing (i.e., to the degree that callus no longer needed to be removed from the explants to recover transgenic plants) and to produce precocious greening and shooting relative to the solid medium, with most of these greening explants expressing the transformation reporter tdTOM and/or GUS (FIG. 3). For example, callus did not need to be removed manually every approximately 3 weeks from each explant.

Reduced level of selection agent in rooting medium. The level of the selection agent spectinomycin included in the ½×Murashige and Skoog (MS)-based rooting medium was titrated back to determine the minimal level at which L1 epidermal events could be separated from germline events (Table 1). Levels as low as 10 mg/L spectinomycin were found to be sufficient to enrich shoots for germline transmission (as determined by either T1 progeny analysis or the presence of transgene in T0 roots). A germline event was obtained using 5 mg/L spectinomycin in the rooting medium, but it was determined that non-transgenic shoots are capable of rooting at this lower level of spectinomycin.

TABLE 1
Germline transformation of Cannabis meristem explants using
different levels of selection during rooting

| | # rooting | # germline | % germline |
|---|---|---|---|
| Spectinomycin | Cannabis plants | events | events |
| 0 mg/L | 22 | 10 | 45% |
| 60.2 mg/L | 7 | 6 | 86% |
| 39 mg/L | 5 | 5 | 100% |
| 10 mg/L | 17 | 17 | 100% |
| 5 mg/L | 1 | 1 | 100% |

Modified rooting medium. A woody plant medium (WPM)-based rooting medium with increased indolebutyric acid (IBA) and no carbenicillin was tested as an alternative to the ½×MS-based rooting medium used in the previous method and was found to enhance the general level and rate of rooting in Cannabis meristem transformation. Further, the new rooting medium was found to rescue shoots obtained using the previous method that had failed to root (i.e., the shoots produced plantlets after being transferred from the old rooting medium to the new rooting medium).

### Protocol Comparison:

A new protocol that includes the simplified excision method, liquid culture, and modified rooting medium discussed above was compared to the Cannabis meristem transformation protocol previously disclosed in U.S. Pat. No. 11,512,320. The new protocol was found to provide enhanced transformation frequency and efficiency (i.e., reduced labor per plant and time to greenhouse) as compared to the previous protocol (Table 2). Specifically, the new protocol was found to dramatically increase the number of transgenic Cannabis plants generated with a given number of explants and to decrease the time from shoot harvest to rooting (Table 3).

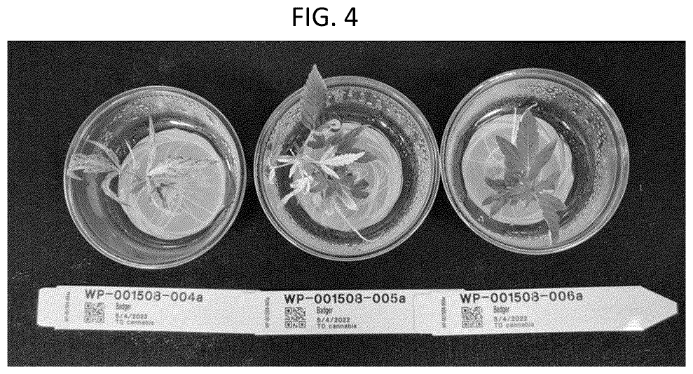

Early variants of the new Cannabis transformation protocol that use liquid medium were tested prior to its full development. Reduced selective pressure in the liquid medium (i.e., 15 mg/L and 25 mg/L spectinomycin) was tested but resulted in no plants, which may have been due to an insufficient advantage of aadA-transformed cells compared with untransformed cells. T0 plants that were initially selected for using 15 mg/L spectinomycin but were transferred to 50 mg/L spectinomycin were recovered, but the resulting plants were occasionally splotchy (i.e., they had bleached regions or spotting but were otherwise green, see FIG. 4), suggesting chimerism. In these initial tests, the older ½×MS-based rooting medium was initially used, and then shoots were transferred to the WPM-based rooting medium where they subsequently rooted. Initial use of the ½×MS-based rooting medium added time to the protocol (i.e., days from inoculation to greenhouse) relative to use of WPM-based rooting medium alone. Some of the plants were rooted on non-selective WPM-based rooting medium, and some likely L1 (non-germline) epidermal events (based on null results in aadA root PCR assays) were sent to greenhouse. These plants were not counted toward total T0 plants or transformation frequency. The transformation metrics for these early iterations of the new protocol are included in the last five rows of Table 2.

TABLE 2
Comparison of transformation metrics for the Cannabis meristem transformation protocol described in U.S. Pat.
No. 11,512,320 (“old”), the improved protocol of the present invention (“new”), and early versions
of the improved protocol that utilized insufficient selection (“early”). In all cases, the data was
generated using the Agrobacterium rhizoenes strain 18r12v (Ar18r12v) and the subcellular location of aadA1a was At_dTP aadA.

| | | | | | | | | | | Average | | Germ- | |
|---|---|---|---|---|---|---|---|---|---|---|---|---|---|
| | | | | | Manual | | | | # | time to | | line | Germ- |
| | Cannabis | Explant | Con- | Selection | callus | Rooting | # | # | T0 | green- | | Lines | line |
| Protocol | variety | excision | struct | medium | removal | medium | Explants | Shoots | plants | house | TF | (T1) | TF |
| Old | Badger | 1 primary | WCIC- | 50 ppm | Every | 10 ppm | 1009 | 11 | 3 | 132 | 0.3% | 2 | 0.2% |
| | | leaf | A-862 | spec | explant, | spec ½X | | | | days | | | |
| | | manually | | SOLID | at each | MS hemp | | | | | | | |
| | | removed | | hemp | subculture | rooting | | | | | | | |
| | | | | node | (every | medium | | | | | | | |
| | | | | medium | 14-21 days) | | | | | | | | |
| New | Badger | primary | WCIC- | 50 ppm | None | 10 ppm | 80 | 4 | 3 | 71 | 3.8% | 3 | 3.8% |
| | | leaves | A-862 | spec | | spec | | | | days | | | |
| | | left intact | | LIQUID | | WPM | | | | | | | |
| | | | | hemp | | hemp | | | | | | | |
| | | | | node | | rooting | | | | | | | |
| | | | | medium | | medium | | | | | | | |
| New | Badger | primary | WCIC- | 50 ppm | None | 10 ppm | 80 | 3 | 3 | 69 | 3.8% | 2 | 2.5% |
| with | | leaves | A-862 | spec | | spec | | | | days | | | |
| GA3 | | left intact | | LIQUID | | WPM | | | | | | | |
| | | | | Hemp | | hemp | | | | | | | |
| | | | | node | | rooting | | | | | | | |
| | | | | medium + | | medium | | | | | | | |
| | | | | 1 ppm | | | | | | | | | |
| | | | | GA3 | | | | | | | | | |
| Old | Badger | 1 primary | WCIC- | 50 ppm | Every | non- | 516 | 5 | 3 | 122 | 0.6% | 3 | 0.6% |
| | | leaf | A-346 | spec | explant, | selective | | | | days | | | |
| | | manually | | SOLID | at each | ½X MS | | | | | | | |
| | | removed | | hemp | subculture | hemp | | | | | | | |
| | | | | node | (every | rooting | | | | | | | |
| | | | | medium | 14-21 days) | medium, | | | | | | | |
| | | | | | | and 5 | | | | | | | |
| | | | | | | ppm spec | | | | | | | |
| | | | | | | WPM | | | | | | | |
| | | | | | | chickpea | | | | | | | |
| | | | | | | BRM* | | | | | | | |
| New | Badger | primary | WCIC- | 50 ppm | None | 10 ppm | 134 | 5 | 2 | 68 | 1.5% | 2 | 1.5% |
| | | leaves | A-346 | spec | | spec | | | | days | | | |
| | | left intact | | LIQUID | | WPM | | | | | | | |
| | | | | hemp | | hemp | | | | | | | |
| | | | | node | | rooting | | | | | | | |
| | | | | medium | | medium | | | | | | | |
| New | Badger | primary | WCIC- | 50 ppm | None | 10 ppm | 246 | 14 | 7 | 60 | 2.8% | 7 | 2.8% |
| | | leaves | A-989 | spec | | spec | | | | days | | | |
| | | left intact | | LIQUID | | WPM | | | | | | | |
| | | | | hemp | | hemp | | | | | | | |
| | | | | node | | rooting | | | | | | | |
| | | | | medium | | medium | | | | | | | |
| New w | Cherry | primary | WCIC- | 50 ppm | None | non- | 44 | 1 | 1 | 54 | 2.3% | 1 | 2.3% |
| non- | Wine | leaves | A-346 | spec | | selective | | | | days | | | |
| selective | Hybrid | left intact | | LIQUID | | WPM | | | | | | | |
| rooting | | | | hemp | | hemp | | | | | | | |
| medium | | | | node | | rooting | | | | | | | |
| | | | | medium | | medium** | | | | | | | |
| New | Badger | primary | DB22 | 50 ppm | None | 10 ppm | 284 | 8 | 4 | 91 | 1.4% | 3 | 1.1% |
| | | leaves | WCIC- | spec | | spec | | | | days | | | |
| | | left intact | A-346 | LIQUID | | WPM | | | | | | | |
| | | | | Hemp | | Hemp | | | | | | | |
| | | | | node | | rooting | | | | | | | |
| Old | Badger | 1 primary | WCIC- | 50 ppm | Every | 10 ppm | 1303 | 5 | 1 | 103 | 0.1% | | |
| | | leaf | A-589 | spec | explant, | spec ½X | | | | days | | | |
| | | manually | | SOLID | at each | MS hemp | | | | | | | |
| | | removed | | hemp | subculture | rooting | | | | | | | |
| | | | | node | (every | medium | | | | | | | |
| | | | | medium | 14-21 days) | | | | | | | | |
| Old | Badger | 1 primary | WCIC- | 50 ppm | Every | 10 ppm | 1560 | 8 | 3 | 255 | 0.2% | | |
| | | leaf | A-590 | spec | explant, | spec ½X | | | | days | | | |
| | | manually | | SOLID | at each | MS hemp | | | | | | | |
| | | removed | | hemp | subculture | rooting | | | | | | | |
| | | | | node | (every | medium | | | | | | | |
| | | | | medium | 14-21 days) | | | | | | | | |
| Old | Badger | 1 primary | WCIC- | 50 ppm | Every | 10 ppm | 870 | 14 | 3 | 169 | 0.3% | | |
| | | leaf | A-734 | spec | explant, | spec ½X | | | | days | | | |
| | | manually | | SOLID | at each | MS hemp | | | | | | | |
| | | removed | | hemp | subculture | rooting | | | | | | | |
| | | | | node | (every | medium | | | | | | | |
| | | | | medium | 14-21 days) | | | | | | | | |
| Early | Badger | 1 primary | WCIC- | 15 ppm | None | 10 ppm | 132 | 1 | 0 | n/a | 0.0% | | |
| | | leaf | A-735 | spec | | spec ½X | | | | | | | |
| | | manually | | LIQUID | | MS hemp | | | | | | | |
| | | removed | | hemp | | rooting | | | | | | | |
| | | | | node | | medium | | | | | | | |
| | | | | medium | | | | | | | | | |
| Early | Badger | 1 primary | WCIC- | 25 ppm | None | 10 ppm | 128 | 2 | 0 | n/a | 0.0% | | |
| | | leaf | A-735 | spec | | spec ½X | | | | | | | |
| | | manually | | LIQUID | | MS hemp | | | | | | | |
| | | removed | | hemp | | rooting | | | | | | | |
| | | | | node | | medium | | | | | | | |
| | | | | medium | | | | | | | | | |
| Early | Badger | 1 primary | WCIC- | 50 ppm | None | 10 ppm | 80 | 1 | 1 | 118 | 1.3% | | |
| | | leaf | A-735 | spec | | spec ½X | | | | days | | | |
| | | manually | | LIQUID | | MS hemp | | | | | | | |
| | | removed | | hemp | | rooting | | | | | | | |
| | | | | node | | medium, | | | | | | | |
| | | | | medium and | | then 10 | | | | | | | |
| | | | | 15 ppm | | ppm spec | | | | | | | |
| | | | | spec | | WPM | | | | | | | |
| | | | | LIQUID | | hemp | | | | | | | |
| | | | | hemp | | rooting | | | | | | | |
| | | | | node | | medium | | | | | | | |
| | | | | medium | | | | | | | | | |
| Early | Badger | primary | WCIC- | 50 ppm | None | 10 ppm | 321 | 11 | 4 | 87 | 1.2% | | |
| | | leaves | A-735 | spec | | spec ½X | | | | days | | | |
| | | left intact | | LIQUID | | MS hemp | | | | | | | |
| | | | | hemp | | rooting | | | | | | | |
| | | | | node | | medium, | | | | | | | |
| | | | | medium and | | then 10 | | | | | | | |
| | | | | 15 ppm | | ppm spec | | | | | | | |
| | | | | spec | | WPM | | | | | | | |
| | | | | LIQUID | | hemp | | | | | | | |
| | | | | hemp | | rooting | | | | | | | |
| | | | | node | | medium | | | | | | | |
| | | | | medium | | | | | | | | | |
| Early | Badger | 1 primary | WCIC- | 50 ppm | None | 10 ppm | 140 | 7 | 3 | 77 | 2.1% | | |
| | | leaf | A-346 | spec | | spec ½X | | | | days | | | |
| | | manually | | LIQUID | | MS Hemp | | | | | | | |
| | | removed | | hemp | | rooting, | | | | | | | |
| | | | | node | | then 10 | | | | | | | |
| | | | | medium and | | ppm spec | | | | | | | |
| | | | | 15-25 ppm | | WPM | | | | | | | |
| | | | | spec | | Hemp | | | | | | | |
| | | | | LIQUID | | rooting | | | | | | | |
| | | | | hemp | | | | | | | | | |
| | | | | node | | | | | | | | | |
| | | | | medium | | | | | | | | | |

TF: transformation frequency.
Germline Lines (T1): Number of T1 germline lines.
Germline TF: transformation frequency of T1 germline lines.
*inclueds confirmed germline 1331-1a, 2a events and 1331-10a (tdTOM positive roots)

includes confirmed germline 1331-1a,2a events and 1331-10a (tdTOM positive roots)

TABLE 3
A breakdown of the average times to greenhouse for
methods described in the first three rows of Table 2

| | Days from | Days from shoot | Total days for |
|---|---|---|---|
| | inoculation to | harvest to | inoculation to |
| Protocol | shoot harvest | rooting | greenhouse |
| Old | 46 | 86 | 132 |
| New | 39 | 32 | 71 |
| New with GA3 | 36 | 33 | 69 |

### T0 Plant Analysis:

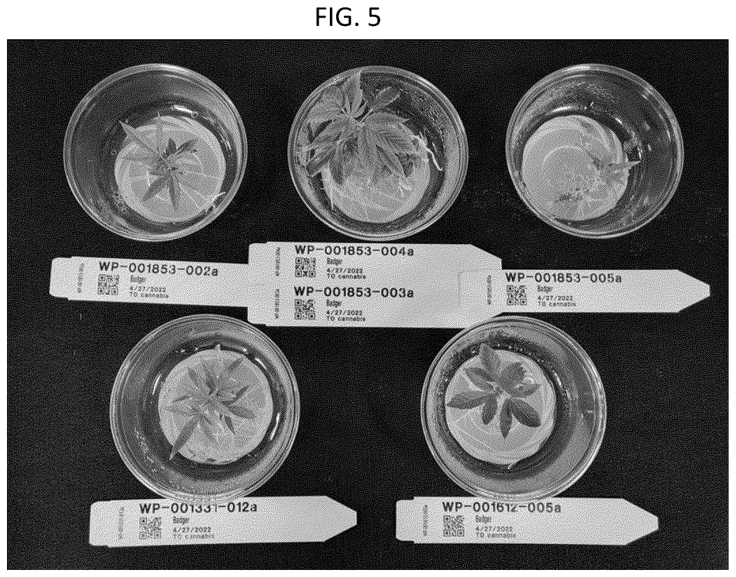

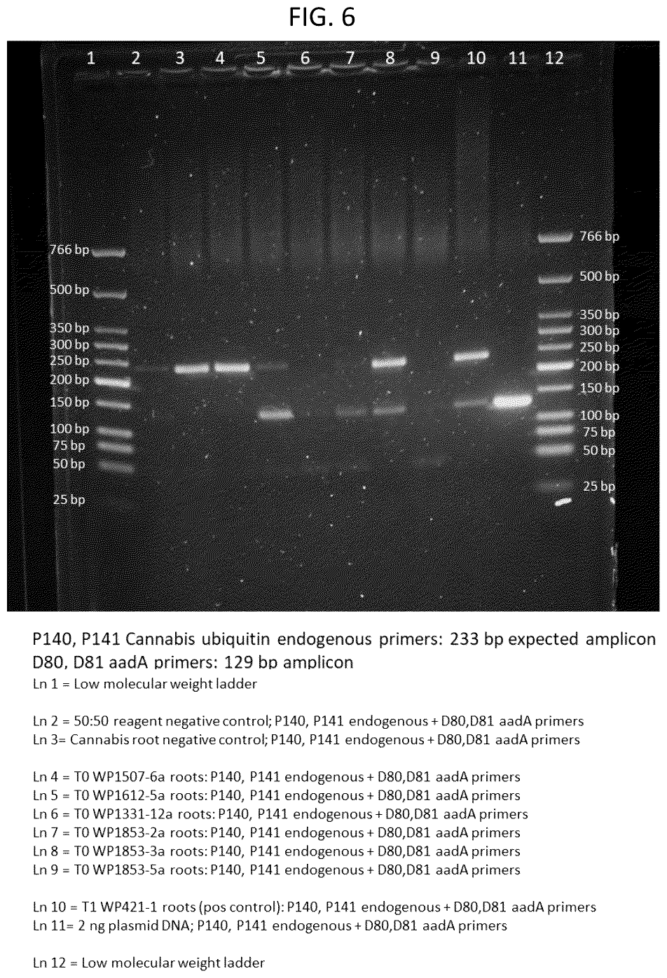

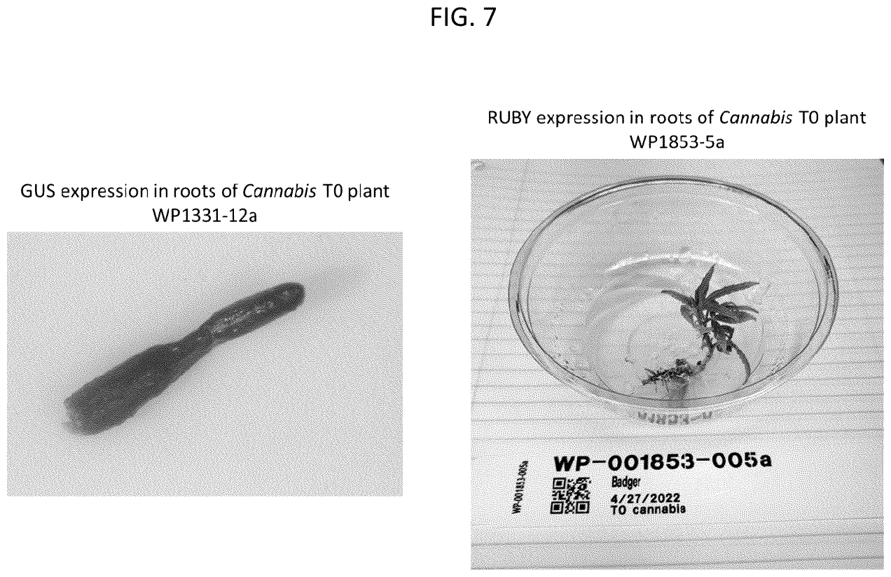

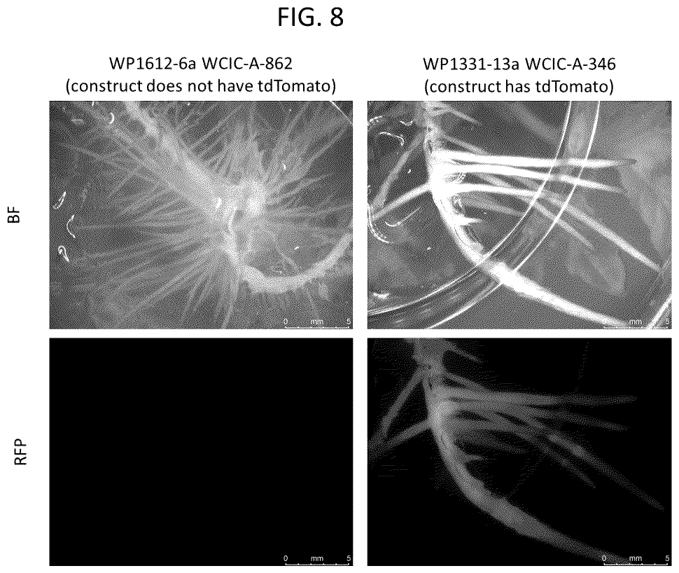

The new Cannabis meristem transformation protocol has been used to generate transformed T0 Cannabis plants (FIG. 5). Multiple assays (i.e., PCR for aadA expression, GUS assay, RUBYv1 expression, and fluorescent detection of tdTOM) have demonstrated that the TO plants are transgene positive in their roots, which is an early indicator of positive germline status (FIG. 6-8, Table 4).

TABLE 4
Putative germline status of Cannabis T0 events generated using
the new Cannabis meristem transformation protocol

| Cannabis | Binary | | |
|---|---|---|---|
| plant ID | construct | Assay | Result |
| WP1612-5a | WCIC-A-862 | PCR for aadA | aadA positive |
| WP1331-12a | WCIC-A-346 | GUS assay | GUS positive |
| WP1853-2a | WCIC-A-989 | PCR for aadA | aadA positive |
| WP1853-3a | WCIC-A-989 | PCR for aadA | aadA positive |
| WP1853-5a,b | WCIC-A-989 | RUBYv1 detection | RUBYvl positive |
| WP1845-1a | WCIC-A-346 | tdTOM detection | tdTOM positive |
| WP1331-13a | WCIC-A-346 | tdTOM detection | tdTOM positive |
| WP1853-6a | WCIC-A-989 | RUBYvl detection | RUBYvl positive |
| WP1612-9a | WCIC-A-862 | PCR for aadA | aadA positive |
| WP1853-7a | WCIC-A-989 | RUBYv1 detection | RUBYv1 positive |
| WP1331-14a | WCIC-A-346 | GUS assay | GUS positive |

### Protocol:

### 1) Cannabis Seed Sanitization and Hydration—Day Before Inoculation:

In a 50 ml centrifuge tube, measure approximately 10 ml of seed.
In a laminar flow hood (LFH), add in 25-30 ml 20% Clorox® to the centrifuge tube, place cap back on tube, place tube on its side (for better seed coverage) and incubate for 5 minutes.
Pour the contents of the tube through an autoclaved strainer, collecting seed in the strainer and allowing Clorox® to collect in a waste bucket. Rinse the seed with ˜500 ml sterile distilled water.
Transfer sanitized seed to an autoclaved beaker. Remove excess liquid with a 1000 μL pipette.
Prepare hydration medium.
   In LFH, add 62.5 L cefotaxime 100 mg/ml stock to 50 ml sterile distilled water (SDW) for a final concentration of 125 mg/L.
   Place 500× Captan/Bravo stock solution (30 mg/ml Captan, 15 mg/ml Bravo in sterile distilled water, with stir bar, stored at 4° C.) on stir plate and allow time for resuspension of the fungicides. Then, in LFH, dilute the stock 500× by adding 100 μL 500× Captan/Bravo stock to 50 ml SDW for a final concentration of 60 mg/L Captan and 30 mg/L Bravo.
In LFH, shake the rehydration medium to resuspend contents and pour over the 5-10 ml sanitized seed. Cover and place at 37° C. in the dark overnight.

### 2) Agrobacterium Preparation-Day Before Inoculation, Early Afternoon:

In LFH, pipette 50 ml LB medium into a 250 ml glass baffle flask.
Most of the WCIC binary constructs use kanamycin as a bacterial selectable marker (stock at 50 mg/ml=50,000 ppm). In LFH, dilute kanamycin stock 1000× in LB by adding 50 μL (of 50 mg/ml stock) to 50 ml LB to for a final concentration of 50 ppm.
Thaw glycerol stock of Ar18r12v comprising the desired binary construct. In LFH, add ˜50 μL of the glycerol stock to the LB+kanamycin.
Grow overnight in shaker set at ˜28 C and ˜200 RPM.
Note: The Agrobacterium cultures can be started at any time, but you will need to add more/less thawed glycerol stock depending on how long they are allowed to grow.

### 3) Explant Excision-Day of Inoculation:

In LFH, rinse imbibed Cannabis seed 3-5× with SDW. Pour into a petri plate for excision. The petri plate may optionally contain a sterile Whatman filter paper to minimize transfer of wounding exudates from the excision step to the final explants.
Remove seed coats with a #11 blade and forceps and place embryos in a fresh dish of SDW.
Rinse embryos 5× with SDW and transfer to a second petri dish with sterile filter paper.
Remove cotyledons from embryo with a #11 blade and forceps under a microscope.
Transfer explants to a fresh dish of SDW. (Grab embryo by the cotyledon to avoid damaging the hypocotyl.)
   About mid-way through this step, spin down and resuspend the Agrobacterium inoculum.
Rinse explants 3-5× with SDW. Limit their “sitting time” in SDW.

### 4) Inoculum Prep-Day of Inoculation:

Check that Agrobacterium has grown (LB should be turbid). In LFH, take a 0.8-1 ml sample and place in a cuvette. Add 0.8-1 ml of LB to separate cuvette to use as a blank. If you don't have a lot of Agrobacterium, you can take 100 μL of your culture and add it to 900 μL LB in your cuvette. The resulting optical density (OD) will be approximately 1/10× of the OD of your culture.
Read the OD660 of the culture in a spectrophotometer by setting wavelength to 660 nm and placing the LB blank in position 1 and the sample in position 2. The OD660 should ideally be between 0.4 and 1.2.
   If the OD660 is low and you need a lot of inoculum, you can put culture back in shaker and let it grow longer.
   If the OD660 is too high (>1.4), dilute your sample 1/10× to make sure the readings are accurate.
   Cells should be harvested in log phase-Inoculum from stationary phase cultures generally appear clumpy and non-uniform.
In LFH, pour the contents of the culture flask into a 50 ml centrifuge tube. Centrifuge for 20 min at 3000 rpm using a H6000A rotor (2619×g) with the brake set at 5.
In LFH, decant supernatant and use 10 ml of dicot INO medium (Table 5) to resuspend the bacterial pellet by pipetting this medium up and down.
In LFH, add additional dicot INO medium to bring target OD660 between 0.3 and 0.4. (Use dicot INO medium as a blank.)
Pour culture back into baffle flask.
   Optionally, add 200 mM acetosyringone stock to a final concentration of 100 μM.
Shake the culture at 23° C. at room temperature and 175 rpm for at least an hour.

### 5) Inoculation and Co-Culture:

Remove water from explants in petri plate. With a sterile scoop or sterile forceps, gently transfer explants to an inverted plantcon. Remove excess liquid with a 1000 ml pipette.
In LFH, add inoculum to explants to cover them (usually at least 25-30 ml per plantcon).
Expose explants to 20 seconds of sonication at 45-55 kHz, then incubate for 30 min on a shaker at ˜75 rpm.
To prepare co-culture medium (Table 6), in LFH, pipette 25 ml dicot INO medium into a 50 ml centrifuge tube or similar sterile container and add:
   25 μl nystatin/thiabendazole (TBZ) stock (50 mg/ml nystatin+10 mg/ml TBZ stock diluted 1000× to 50 ppm and 10 ppm, respectively), and
   25 μL thidiazuron (TDZ) stock (1 mg/ml TDZ stock diluted 1000× to 1 ppm).
   Note: TDZ inhibits native plant cytokinin oxidase and acts as a cytokinin. Both encourage growth/budding in secondary meristems and reduce germination response in SAM.
In LFH, remove excess inoculum to remove un-attached Agrobacterium.
Optionally, rinse the explants 1× with SDW and fully remove water.
In LFH, prepare co-culture plantcons by placing a square piece of sterile filter paper in plantcon bottoms and pipette co-culture medium onto the filter paper.
   Note: Different amounts of co-culture medium can be used. For example, 1.25 ml, 1.5 ml, 1.75 ml, 2.25 ml, and 2.5 ml have all been used successfully.
In LFH, move explants onto filter papers with sterile forceps (˜35 explants per plankton).
Place co-culture plantcons in a 23° C. 16 h light/8 h dark photoperiod Percival for 4 days.

### 6) Selection:

Optional 3 day delay prior to transferring explants to liquid selection medium.
In LFH, transfer explants to liquid selection medium.
   Prepare filter beds by placing 4 sterile 8.2 cm filter papers in a deep-dish petri plate. Add 15 ml liquid selection medium, i.e., hemp node medium supplemented with 50 mg/L active spectinomycin, 0.5 mg/L meta-topolin, 250 mg/L carbenicillin, 200 mg/L cefotaxime, and 150 mg/L timetin (Table 7).
   Transfer 16-20 explants to each plate.
Wrap plate with venting tape.
Place in a 27° C. 16/8 photoperiod Percival.
Every ˜10 days, add 10 ml fresh liquid hemp node medium (with the same additives) to each plate. When humidity is low, you may need to do this about every 7 days.
Optionally, transfer greening phenotypes (spectinomycin will bleach the non-transformed cells) to a fresh filter bed with 15 ml liquid hemp node medium (with the same additives) as they develop.

### 7) Shoot Harvest and Rooting:

Harvest greening shoots that are at least 0.5 cm long. Use a microscope to check the health of the shoots and determine where to cut them (want to cut at a node to increase rooting potential).
Use a #11 blade to embed shoots into rooting medium, i.e., WPM hemp rooting medium comprising 10 mg/L active spectinomycin and 5× indole-3-butyric acid (IBA), without carbenicillin (Table 8).
Place shoots in a 27° C. 16/8 photoperiod Percival for about 4-8 weeks.
Transfer shoots to the greenhouse once they have developed decent roots (e.g., at least 2 cm long) if their leaves are still green.

### Media Used in Protocol:

TABLE 5
dicot INO medium

| Ingredients and notes | Amount to add per liter | |
|---|---|---|
| Phytotechnology Laboratories B5 salts G398 | 1.284 | g |
| Glucose | 30 | g |
| MES hydrate (Alfa Aesar CAS 4432-31-9) | 2.8 | g |
| pH to 5.4 with IN KOH | | |
| autoclave | | |

TABLE 6
Co-culture medium: dicot INO medium with TBZ and TDZ

| Ingredients and notes | Amount | |
|---|---|---|
| Dicot INO medium | 1 | L |
| Nystatin/thiabendazole (TBZ) (50 mg/ml | 1 | ml |
| nystatin and 10 mg/ml TBZ stock) | | |
| Thidiazuron (TDZ) (1 mg/ml stock) | 1 | ml |

TABLE 7
Selection medium: liquid hemp node regeneration medium

| Ingredients and notes | Amount to add per liter | |
|---|---|---|
| MS Salts complete with vitamins | 4.43 | g |
| (PhytoTech M519) | | |
| Sucrose | 30 | g |
| Cleary's 3336 | 0.06 | g |
| pH to 5.7 with IN KOH | | |
| autoclave | | |
| Meta-topolin (mT) (1 mg/ml) | 0.5 | ml |
| Carbenicillin (100 mg/ml) | 2.5 | ml |
| Cefotaxime (100 mg/ml) | 2 | ml |
| Timetin (150 mg/ml) | 1 | ml |

| | |
|---|---|
| Selection | as needed |

TABLE 8
Rooting medium: WPM hemp rooting medium with 5X IBA,
without carbenicillin

| Ingredients and Notes | Amount to add per liter | |
|---|---|---|
| WPM salts (Phytotechnology | 2.41 | g |
| Laboratories WPM L449) | | |
| Sucrose | 15 | g |
| pH to 5.6 with KOH | | |
| Agar (Sigma A7921) | 8 | g |
| Autoclave | | |
| IBA stock (1 mg/ml) | 2.55 | ml |
| Cefotaxime (100 mg/ml stock) | 2 | ml |
| Timentin (150 mg/ml stock) | 1 | ml |
| Selection | as needed | |
| Container | ice cream dishes | |
| Distribution | ~35 | ml/dish |

### Example 2

In the following example, the inventors describe several methods that are hybrids of the new Cannabis transformation protocol described herein and the previous protocol described in U.S. Pat. No. 11,512,320. These hybrid methods were assessed as part of the development of the new protocol. Table 9 outlines the major differences between the old, hybrid, and new protocols. Table 10 details the generation of T0 Cannabis plants using a variety of protocols, which are as classified as “old,” “hybrid,” or “new” based on the following criteria:
Old:
   One primary leaf of the explant was left intact
   Shoots were developed on solid selection medium
   T0 plants were sent to the greenhouse as rooted hypocotyls or rooted shoots on ½×MS-based medium
Hybrid:
   One or both primary leaves of the explant were left intact
   Shoots were developed on solid or liquid selection medium
   T0 plants were sent to the greenhouse as rooted shoots on ½×MS- or WPM-based medium
New:
   Both primary leaves of the explant were left intact
   Shoots were developed on liquid selection medium
   T0 plants were sent to the greenhouse as rooted shoots on WPM-based medium

TABLE 9
Comparison of the old, new, and hybrid Cannabis transformation protocols

| | Old protocol | Hybrid protocols | |
|---|---|---|---|
| | 5 min in 20% Clorox, or 1 min | 5 min in 20% Clorox, or 1 min | |
| Protocol step | in 70% EtOH followed by 5 | in 70% EtOH followed by 5 | New protocol |
| Seed sanitization | min in 20% Clorox | min in 20% Clorox | 5 min in 20% Clorox |
| Prime step (interval | ~2 hours | ~2 hours or none | none |
| between seed | | | |
| sanitization and | | | |
| imbibition) | | | |
| Imbibition medium | WCIC BGM | WCIC BGM or SDW | SDW |
| Imbibition time | overnight | overnight | overnight |
| Imbibition | 23° C. or 37° C. | 23° C.- 37° C. | 37° C. |
| temperature | | | |
| Preculture | None (Agrobacterium) or | None or EJW1 | None |
| | EJW1 (particle bombardment) | (Agrobacterium) | |
| Selection | aadA (spectinomycin and | aadA (spectinomycin) | aadA (spectinomycin) |
| | streptomycin) | | |
| Selection targeting | CTP | CTP, At dTP | At dTP |
| Primary leaves | At least one primary leaf | At least one primary leaf | Retained |
| | removed (Agrobacterium) or | removed (Agrobacterium) | |
| | both primary leaves removed | | |
| | (particle gun) | | |
| Co-culture | 3-5 d 23° C. 16/8 photoperiod | 3-5 d 23° C. 16/8 photoperiod | 3-5 d 23° C. 16/8 |
| | | or dark | photoperiod |
| Selection / | Solid MS-based medium with | Solid or liquid MS-based | Liquid MS-based |
| regeneration | spectinomycin | medium with spectinomycin | medium with |
| medium for shoot | | | spectinomycin |
| development | | | |
| Transfer regime | Explants transferred to fresh | Explants transferred to fresh | No callus removal; |
| | medium every 2-4 weeks with | medium every 2-4 weeks with | ~10 ml fresh medium |
| | manual removal of callus from | manual removal of callus from | added every ~10 days |
| | each explant hypocoty1 | each explant hypocoty1 | |
| T0 sent to | Rooted hypocotyl or rooted | Rooted shoot | Rooted shoot |
| greenhouse | shoot | | |
| Medium used for | Solid MS-based medium with | Solid 1/2 MS-based medium; | Solid WPM-based |
| root development | either streptomycin or non- | solid WPM-based medium | medium with |
| | selective (hypocotyl); or solid | with spectinomycin | spectinomycin |
| | 1/2 MS-based medium (shoot) | | |
| Selection level | 150 mg/L streptomycin or 0- | 0-62.5 mg/L spectinomycin | 10 mg/L |
| during root | 62.5 mg/L spectinomycin | | spectinomycin |
| development | | | |

TABLE 10
Comparison of various protocols used to generate of T0 Cannabis plants

| | | | | | | | | | | T1 segregation |
|---|---|---|---|---|---|---|---|---|---|---|
| | | | | | | | | | | (1:1 expected |
| | | | | | | | | | | for non- |
| | | | Can- | | | | | | | chimeric |
| | | | nabis | | | | Whole | | T0 GUS/ | single copy |
| Event | Pro- | Con- | Geno- | Seed | Co- | | explant/ | Rooting | tdTOM/ | event crossed |
| ID | tocol | struct | type | conditions | culture | Regeneration | Shoot | medium | PCR | with wild type) |
| WP421- | Old | DB19 | Cherry | 5 min 20% | 2.5 ml co- | 4 weeks | Whole | 50 mg/L | 10/10 leaves | Germline |
| 1 | | | Wine | Clorox; 23° C. | culture; 4 d | 50 mg/L | explant | streptomycin | GUS+, | segregating |
| | | | Hybrid | imbibition in | 23 C. 16/8 | active | | Hemp node | tdTOM+, | 1:1 |
| | | | (3WS) | BGM | photoperiod | spectinomycin | | | CTP+ by | |
| | | | | | | Hemp Node | | | PCR | |
| WP421- | Old | DB19 | Cherry | 5 min 20% | 2.5 ml co- | 3 weeks | Whole | 50 mg/L | Initial | Event |
| 2 | | | Wine | Clorox; 23° C. | culture; 4 d | 75 mg/L | explant | streptomycin | leaves | tossed |
| | | | Hybrid | imbibition in | 23 C. 16/8 | active | | Hemp node | chimeric | (escape) |
| | | | (3WS) | BGM | photoperiod | spectinomycin | | without mT; | GUS | |
| | | | | | | Hemp Node | | then Soy | positive, | |
| | | | | | | | | BRM (½X | but not | |
| | | | | | | | | MS) without | detected | |
| | | | | | | | | selection | later in | |
| | | | | | | | | | GH plant | |
| WP1182- | Old | DICOT | Cherry | 5 min 20% | n/a | 4 weeks | Whole | 50 mg/L | 10/10 | Epidermal; |
| 1a | | BOMB13 | Wine | Clorox; 37° C. | PARTICLE | 100 mg/L | explant | streptomycin | leaves | 0 POS; 6 |
| | | | Hybrid | imbibition in | GUN; | active | | hemp node; | GUS+; | null |
| | | | | BGM; | 1.2 ng | spectinomycin | | then Soy | CTP+ by | |
| | | | | explant | DNA/ug | Hemp Node | | BRM | PCR | |
| | | | | precultured | 0.6 um | | | without | | |
| | | | | on EJW1 o/n | gold DLR | | | selection | | |
| | | | | at 27° C. 16/8 | | | | | | |
| | | | | photoperiod | | | | | | |
| WP1182- | Old | DICOT | Cherry | 5 min 20% | n/a | 4 weeks | Whole | 50 mg/L | 10/10 | No T1 seed |
| 1b | | BOMB13 | Wine | Clorox; 37° C. | PARTICLE | 100 mg/L | explant | streptomycin | leaves | set (We |
| | | | Hybrid | imbibition in | GUN; | active | | hemp node; | GUS+; | attempted to |
| | | | | BGM; | 1.2 ng | spectinomycin | | then Soy | CTP+ by | masculinize |
| | | | | explant | DNA/ug | Hemp Node | | BRM | PCR | this clone |
| | | | | precultured | 0.6 um | | | without | | to pollinate |
| | | | | on EJW1 o/n | gold DLR | | | selection | | the -1a |
| | | | | at 27° C. 16/8 | | | | | | event to |
| | | | | photoperiod | | | | | | produce |
| | | | | | | | | | | homozygotes) |
| WP1331- | Old | DB22 | Badger | 5 min 20% | 1.5 ml co- | 6 weeks 50 | Rooted | 5 mg/L | Chimeric; | Germline; |
| 1a | | | | Clorox; 37° C. | culture; 5 d | mg/L | shoot | active | 3/5 | 3 POS: 8 |
| | | | | imbibition in | 23 C. dark | active | | spectinomycin | leaves | null |
| | | | | BGM | | spectinomycin | | Chickpea BRM | aadA+ | |
| | | | | | | Hemp Node | | (WPM BRM) | by PCR | |
| WP1331- | Old | DB22 | Badger | 5 min 20% | 1.25 ml | 6 weeks 50 | Rooted | 5 mg/L | Uniform | Germline; |
| 2a | | | | Clorox; 37° C. | co-culture; | mg/L | shoot | active | tdTOM+ | 9 POS; 6 |
| | | | | imbibition in | 5 d 23 C. | active | | spectinomycin | throughout | null |
| | | | | BGM | dark | spectinomycin | | Chickpea | plant | |
| | | | | | | Hemp Node | | BRM; then | | |
| | | | | | | | | Hemp rooting | | |
| | | | | | | | | medium | | |
| | | | | | | | | without | | |
| | | | | | | | | selection | | |
| WP1300- | Old | DB19 | Badger | 122° F. heat | Approximately | 12 weeks | Rooted | 39 mg/L | | Germline: |
| 2a | | | | for 20 min; | 2.5 ml co- | 60.2 mg/L | shoot | active | | 4 POS; 4 |
| | | | | 37° C. | culture; 4 d | active | | spectinomycin | | null |
| | | | | imbibition in | 23 C. 16/8 | spectinomycin | | Hemp | | |
| | | | | H2O | photoperiod | Hemp Node | | Rooting with | | |
| | | | | | | | | 500 mg/L | | |
| | | | | | | | | activated | | |
| | | | | | | | | charcoal | | |
| WP1319- | Old | DB19 | Badger | 5 min 20% | Rinsed 1X | 3 weeks 30 | Rooted | 60.2 mg/L | Leaf | Germline: |
| 1a | | | | Clorox then | post | mg/L | shoot | active | sample | 3 POS; 8 |
| | | | | 37° C. | inoculation; | active | | spectinomycin | tdTOM+, | null |
| | | | | imbibition in | Approximately | spectinomycin | | Hemp | GUS+, | |
| | | | | BGM, OR | 2.5 ml co- | ½X MS | | Rooting | and | |
| | | | | 122° F. for 20 | culture; 4 d | Hemp Node; | | | aadA+ | |
| | | | | min, SDW, | 23 C. 16/8 | 2 weeks on | | | by PCR | |
| | | | | 37° C. | photoperiod | 60.2 mg/L | | | | |
| | | | | overnight | | active | | | | |
| | | | | | | spectinomycin | | | | |
| | | | | | | Hemp Node; | | | | |
| | | | | | | 2.5 weeks | | | | |
| | | | | | | 60.2 mg/L | | | | |
| | | | | | | active | | | | |
| | | | | | | spectinomycin | | | | |
| | | | | | | WPM | | | | |
| WP1345- | Old | WCIC- | Badger | 5 min 20% | Rinsed 1X | 6 weeks | Rooted | 60.2 mg/L | 4/4 | Germline: |
| 1a | | A-591 | | Clorox; 37° C. | post | 60.2 mg/L | shoot | active | leaves | 5 POS; 3 |
| | | | | imbibition in | inoculation; | active | | spectinomycin | aadA+ | null |
| | | | | BGM | Approximately | spectinomycin | | Hemp Rooting | by PCR | |
| | | | | | 2.5 ml co- | Hemp Node | | | | |
| | | | | | culture; 4 d | | | | | |
| | | | | | 23 C. 16/8 | | | | | |
| | | | | | photoperiod | | | | | |
| WP1345- | Old | WCIC- | Badger | 1 min 100% | Rinsed 1X | 6 weeks | Rooted | 60.2 mg/L | 3/5 | Germline: |
| 2a | | A-591 | | EtOH, then 5 | post | 60.2 mg/L | shoot | active | leaves | 1 POS; 3 |
| | | | | min 20% | inoculation; | active | | spectinomycin | aadA+ | null |
| | | | | Clorox; 37° C. | Approximately | spectinomycin | | Hemp Rooting | by PCR | |
| | | | | imbibition in | 2.5 ml co- | Hemp Node | | | | |
| | | | | SDW | culture; 4 d | | | | | |
| | | | | | 23 C. 16/8 | | | | | |
| | | | | | photoperiod | | | | | |
| WP1345- | Old | WCIC- | Badger | 1 min 100% | Rinsed 1X | 12 weeks | Rooted | 60.2 mg/L | 3/4 | Germline: |
| 3a | | A-591 | | EtOH, then 5 | post | 60.2 mg/L | shoot | active | leaves | 8 POS, 9 |
| | | | | min 20% | inoculation; | active | | spectinomycin | aadA+ | null |
| | | | | Clorox; 37° C. | Approximately | spectinomycin | | Hemp Rooting | by PCR | |
| | | | | imbibition in | 2.5 ml co- | Hemp Node | | | | |
| | | | | SDW | culture; 4 d | | | | | |
| | | | | | 23 C. 16/8 | | | | | |
| | | | | | photoperiod | | | | | |
| WP1345- | Old | WCIC- | Badger | 1 min 100% | Rinsed 1X | 8 weeks | Rooted | 39 mg/L | 2/3 | Germline: |
| 4a | | A-591 | | EtOH, then 5 | post | 60.2 mg/L | shoot | active | leaves | 4 POS; 6 |
| | | | | min 20% | inoculation; | active | | spectinomycin | aadA+ | null |
| | | | | Clorox; 37° C. | Approximately | spectinomycin | | Hemp Rooting | by PCR | |
| | | | | imbibition in | 2.5 ml co- | Hemp Node | | | | |
| | | | | SDW | culture; 4 d | | | | | |
| | | | | | 23 C. 16/8 | | | | | |
| | | | | | photoperiod | | | | | |
| WP1345- | Old | WCIC- | Badger | 5 min 20% | 1.5 ml co- | 5 weeks | Rooted | 50 mg/L | 2/2 | Germline: |
| 5a | | A-591 | | Clorox; 37° C. | culture; 4 d | 50 mg/L | shoot | active | leaves | 2 POS; 4 |
| | | | | imbibition in | 23 C. 16/8 | active | | spectinomycin | aadA+ | null |
| | | | | BGM | photoperiod | spectinomycin | | Hemp rooting | by PCR | |
| | | | | | | Hemp Node | | medium; | (3^{rd} leaf | |
| | | | | | | (surface | | then 10 mg/L | no | |
| | | | | | | plated) | | active | endogenous | |
| | | | | | | | | spectinomycin | band) | |
| | | | | | | | | Hemp rooting | | |
| | | | | | | | | medium | | |
| WP1345- | Old | WCIC- | Badger | 1 min 100% | Rinsed 1X | 8 weeks | Rooted | 39 mg/L | 4/4 | Germline: |
| 6a | | A-591 | | EtOH, then 5 | post | 60.2 mg/L | shoot | active | leaves | 2 POS; 1 |
| | | | | min 20% | inoculation; | active | | spectinomycin | aadA+ | null |
| | | | | Clorox; 37° C. | Approximately | spectinomycin | | Hemp Rooting | by PCR | |
| | | | | imbibition in | 2.5 ml co- | Hemp Node | | with 500 mg/L | | |
| | | | | SDW | culture; 4 d | | | activated | | |
| | | | | | 23 C. 16/8 | | | charcoal | | |
| | | | | | photoperiod | | | | | |
| WP1346- | Old | WCIC- | Badger | 5 min 20% | Rinsed 1X | 6 weeks | Rooted | 60.2 active | Autoflowering; | Epidermal |
| 1a | | A-592 | | Clorox; 37° C. | post | 60.2 mg/L | shoot | spectinomycin | endogenous | (cuttling |
| | | | | imbibition in | inoculation; | active | | Hemp Rooting | bands did | likely |
| | | | | BGM | Approximately | spectinomycin | | | not amplify | negative): |
| | | | | | 2.5 ml co- | Hemp Node | | | in PCR; | 0 POS, 16 |
| | | | | | culture; 4 d | | | | cutting of | null |
| | | | | | 23 C. 16/8 | | | | this plant | |
| | | | | | photoperiod | | | | gave 0/4 | |
| | | | | | | | | | leaves | |
| | | | | | | | | | aadA+ | |
| | | | | | | | | | by PCR | |
| WP1346- | Old | WCIC- | Badger | 1 min 100% | Rinsed 1X | 9 weeks | Rooted | 60.2 active | 4/4 | Germline: |
| 2a | | A-592 | | EtOH, then 5 | post | 60.2 mg/L | shoot | spectinomycin | leaves | 1 POS; 5 |
| | | | | min 20% | inoculation; | active | | Hemp Rooting | aadA+ | null |
| | | | | Clorox; 37° C. | Approximately | spectinomycin | | | by PCR | |
| | | | | imbibition in | 2.5 ml co- | Hemp Node | | | | |
| | | | | SDW | culture; 4 d | | | | | |
| | | | | | 23 C. 16/8 | | | | | |
| | | | | | photoperiod | | | | | |
| WP1346- | Old | WCIC- | Badger | 1 min 100% | Rinsed 1X | 9 weeks | Rooted | 60.2 active | 4/4 | Germline: |
| 3a | | A-592 | | EtOH, then 5 | post | 60.2 mg/L | shoot | spectinomycin | leaves | 4 POS; 3 |
| | | | | min 20% | inoculation; | active | | Hemp Rooting | aadA+ | null |
| | | | | Clorox; 37° C. | Approximately | spectinomycin | | | by PCR | |
| | | | | imbibition in | 2.5 ml co- | Hemp Node | | | | |
| | | | | SDW | culture; 4 d | | | | | |
| | | | | | 23 C. 16/8 | | | | | |
| | | | | | photoperiod | | | | | |
| WP1346- | Old | WCIC- | Badger | 1 min 100% | Rinsed 1X | 8.5 weeks | Rooted | 39 mg/L | 4/4 | Germline: |
| 4a | | A-592 | | EtOH, then 5 | post | 60.2 mg/L | shoot | active | leaves | 4 POS; 1 |
| | | | | min 20% | inoculation; | active | | spectinomycin | aadA+ | null |
| | | | | Clorox; 37° C. | Approximately | spectinomycin | | Hemp Rooting | by PCR | |
| | | | | imbibition in | 2.5 ml co- | Hemp Node | | with 500 mg/L | | |
| | | | | SDW | culture; 4 d | | | activated | | |
| | | | | | 23 C. 16/8 | | | charcoal | | |
| | | | | | photoperiod | | | | | |
| WP1530- | Old | WCIC- | Cherry | 5 min 20% | Rinsed 1X | 7 weeks | Rooted | 39 mg/L | 4/4 | Germline: |
| 1a | | A-591 | Wine | Clorox; 37° C. | post | 60.2 mg/L | shoot | active | leaves | 1 POS; 2 |
| | | | Hybrid | imbibition in | inoculation; | active | | spectinomycin | aadA+ | null |
| | | | | BGM | Approximately | spectinomycin | | Hemp Rooting | by PCR | |
| | | | | | 2.5 ml co- | Hemp Node | | with 500 mg/L | | |
| | | | | | culture; 5 d | | | activated | | |
| | | | | | 23 C. 16/8 | | | charcoal | | |
| | | | | | photoperiod | | | | | |
| WP1344- | Old | WCIC- | Badger | 5 min 20% | Rinsed 1X | 5 weeks 50 | Rooted | 10 mg/L | Died in | |
| 1a | | A-590 | | Clorox; 37° C. | post | mg/L | shoot | active | GH | |
| | | | | imbibition in | inoculation; | active | | spectinomycin | nursery | |
| | | | | SDW | 2.25 ml | spectinomycin | | Hemp rooting | | |
| | | | | | co-culture; | Hemp Node | | medium | | |
| | | | | | 4 d 16/8 | | | | | |
| | | | | | photoperiod | | | | | |
| WP1344- | Old | WCIC- | Badger | 1 min 100% | 2 ml co- | 7 weeks | Rooted | 10 mg/L | small | |
| 2a | | A-590 | | EtOH, then 5 | culture; 4 d | 50 mg/L | shoot | active | branch | |
| | | | | min 20% | 16/8 | active | | spectinomycin | has 3/3+ | |
| | | | | Clorox; 37° C. | photoperiod | spectinomycin | | Hemp rooting | leaves | |
| | | | | imbibition in | | Hemp Node | | medium | aadA by | |
| | | | | SDW | | | | | PCR; | |
| | | | | | | | | | large | |
| | | | | | | | | | branch | |
| | | | | | | | | | had 0/3+ | |
| | | | | | | | | | aadA leaves | |
| | | | | | | | | | and was | |
| | | | | | | | | | pruned | |
| WP1345- | Hy- | WCIC- | Badger | 5 min 20% | 1.75 ml | 10 weeks | Rooted | 50 mg/L | Branching | |
| 11a | brid | A-591 | | Clorox; 37° C. | co-culture; | 50 mg/L | shoot | active | plant; | |
| | | | | imbibition in | 3 d 16/8 | active | | spectinomycin | both | |
| | | | | BGM; | photoperiod | spectinomycin | | Hemp rooting | branches 3/3 | |
| | | | | explants then | | Hemp Node | | medium; | leaves | |
| | | | | precultured | | | | then 10 mg/L | aadA+ | |
| | | | | on EJW1 | | | | active | by PCR | |
| | | | | 37° C. dark | | | | spectinomycin | | |
| | | | | o/n | | | | Hemp rooting | | |
| | | | | | | | | medium | | |
| WP1345- | Old | WCIC- | Badger | 5 min 20% | 2.5 ml co- | 5 weeks | Rooted | 50 mg/L | | |
| 12a | | A-591 | | Clorox; 37° C. | culture; 4 d | 50 mg/L | shoot | active | | |
| | | | | imbibition in | 16/8 | active | | spectinomycin | | |
| | | | | BGM | photoperiod | spectinomycin | | Hemp rooting | | |
| | | | | | | modified | | medium; | | |
| | | | | | | Hemp Node | | then 10 mg/L | | |
| | | | | | | (premix | | active | | |
| | | | | | | MS salts | | spectinomycin | | |
| | | | | | | with B5 | | Hemp rooting | | |
| | | | | | | vitamins; | | medium | | |
| | | | | | | phytagel | | | | |
| | | | | | | replacing | | | | |
| | | | | | | agar) | | | | |
| WP1502- | Old | WCIC- | Badger | 5 min 20% | Rinsed 1X | 6 weeks | Rooted | 10 mg/L | | |
| 1a | | A-589 | | Clorox; 37° C. | post | 50 mg/L | shoot | active | | |
| | | | | imbibition in | inoculation; | active | | spectinomycin | | |
| | | | | SDW | 2 ml co- | spectinomycin | | Hemp rooting | | |
| | | | | | culture; 5 d | Hemp Node | | medium | | |
| | | | | | 16/8 | | | | | |
| | | | | | photoperiod | | | | | |
| WP1507- | Old | WCIC- | Badger | 1 min 100% | Rinsed 1X | 5 weeks | Rooted | 10 mg/L | Plant | |
| 1a | | A-734 | | EtOH, then 5 | post | 50 mg/L | shoot | active | died in | |
| | | | | min 20% | inoculation; | active | | spectinomycin | nursery | |
| | | | | Clorox; 37° C. | 2 ml co- | spectinomycin | | Hemp rooting | | |
| | | | | imbibition in | culture; 5 d | Hemp Node | | medium | | |
| | | | | SDW | 16/8 | | | | | |
| | | | | | photoperiod | | | | | |
| WP1507- | Old | WCIC- | Badger | 5 min 20% | Rinsed 1X | 5 weeks | Rooted | 10 mg/L | | |
| 2a | | A-734 | | Clorox; 37° C. | post | 50 mg/L | shoot | active | | |
| | | | | imbibition in | inoculation; | active | | spectinomycin | | |
| | | | | SDW | 2 ml co- | spectinomycin | | Hemp Rooting | | |
| | | | | | culture; 5 d | Hemp Node | | | | |
| | | | | | 16/8 | | | | | |
| | | | | | photoperiod | | | | | |
| WP1331- | Hy- | DB22 | Badger | 1 min 100% | 2.25 ml | 3.5 weeks | Rooted | Non- | Leaves | |
| 3a | brid | | | EtOH, then 5 | co-culture; | 50 mg/L | shoot | selective | aadA+ | |
| | | | | min 20% | 4 d 16/8 | active | | Hemp | by PCR | |
| | | | | Clorox; 37° C. | photoperiod | spectinomycin | | Rooting | and | |
| | | | | imbibition in | | LIQUID | | minus carb | GUS+; | |
| | | | | SDW | | Hemp Node | | with 5X IBA | roots | |
| | | | | | | | | | aadA | |
| | | | | | | | | | negative | |
| | | | | | | | | | by PCR | |
| | | | | | | | | | and GUS | |
| | | | | | | | | | negative; | |
| | | | | | | | | | putative | |
| | | | | | | | | | epidermal | |
| WP1593- | Old | WCIC- | Badger | 5 min 20% | Rinsed 1X | 8 weeks | Rooted | Non- | Leaves | |
| 1a | | A-730 | | Clorox; 37° C. | post | 50 mg/L | shoot | selective | aadA+ | |
| | | | | imbibition in | inoculation; | active | | Hemp Rooting | by PCR | |
| | | | | SDW | 2.25 ml | spectinomycin | | minus carb | and roots | |
| | | | | | co-culture; | Hemp Node | | with 5X IBA | aadA | |
| | | | | | 5 d 16/8 | | | | negative | |
| | | | | | photoperiod | | | | by PCR; | |
| | | | | | | | | | putative | |
| | | | | | | | | | epidermal | |
| WP1331- | Hy- | DB22 | Badger | 5 min 20% | Rinsed 1X | 4 weeks | Rooted | 10 mg/L | Leaves | |
| 4a | brid | | | Clorox; 37° C. | post | 75 mg/L | shoot | active | and roots | |
| | | | | imbibition in | inoculation; | active | | spectinomycin | tdTOM; | |
| | | | | SDW | 2.25 ml | spectinomycin | | Hemp Rooting | leaves | |
| | | | | | co-culture; | LIQUID | | minus carb | aadA+ | |
| | | | | | 4 d 16/8 | Hemp Node | | | by PCR; | |
| | | | | | photoperiod | | | | putative | |
| | | | | | | | | | germline | |
| WP1612- | Old | WCIC- | Badger | 1 min 100% | Rinsed 1X | 8 weeks | Rooted | 10 mg/L | Leaves | |
| 1a | | A-862 | | EtOH, then 5 | post | 50 mg/L | shoot | active | and roots | |
| | | | | min 20% | inoculation; | active | | spectinomycin | aadA+ | |
| | | | | Clorox; 37° C. | 2.25 ml | spectinomycin | | Hemp Rooting | by PCR; | |
| | | | | imbibition in | co-culture; | Hemp Node | | | Putative | |
| | | | | SDW | 4 d 16/8 | | | | germline | |
| | | | | | photoperiod | | | | | |
| WP1331- | Hy- | DB22 | Badger | 5 min 20% | Rinsed 1X | 4 weeks | Rooted | Non- | Roots | |
| 5a | brid | | | Clorox; 37° C. | post | 50 mg/L | shoot | selective | tdTOM | |
| | | | | imbibition in | inoculation; | active | | Hemp Rooting | positive; | |
| | | | | SDW | 2.25 ml | spectinomycin | | minus carb | Putative | |
| | | | | | co-culture; | LIQUID | | with 5X IBA | germline | |
| | | | | | 4 d 16/8 | Hemp Node | | | | |
| | | | | | photoperiod | | | | | |
| WP1331- | Hy- | DB22 | Badger | 5 min 20% | Rinsed 1X | 4 weeks | Rooted | Non- | Roots | |
| 5b | brid | | | Clorox; 37° C. | post | 50 mg/L | shoot | selective | tdTOM | |
| | | | | imbibition in | inoculation; | active | | Hemp Rooting | positive; | |
| | | | | SDW | 2.25 ml | spectinomycin | | minus carb | Putative | |
| | | | | | co-culture; | LIQUID | | with 5X IBA | germline | |
| | | | | | 4 d 16/8 | Hemp Node | | | | |
| | | | | | photoperiod | | | | | |
| WP1331- | Hy- | DB22 | Badger | 5 min 20% | Rinsed 1X | 4 weeks | Rooted | Non- | Roots | |
| 6a | brid | | | Clorox; 37° C. | post | 50 mg/L | shoot | selective | tdTOM | |
| | | | | imbibition in | inoculation; | active | | Hemp Rooting | negative; | |
| | | | | SDW | 2.25 ml | spectinomycin | | minus carb | Putative | |
| | | | | | co-culture; | LIQUID | | with 5X IBA | epidermal | |
| | | | | | 4 d 16/8 | Hemp Node | | | | |
| | | | | | photoperiod | | | | | |
| WP1507- | Old | WCIC- | Badger | 5 min 20% | Rinsed 1X | 8 weeks | Rooted | Non- | Roots | |
| 3a | | A-734 | | Clorox; 37° C. | post | 50 mg/L | shoot | selective | aadA | |
| | | | | imbibition in | inoculation; | active | | Hemp | negative | |
| | | | | SDW | 2 ml co- | spectinomycin | | Rooting with | by PCR; | |
| | | | | | culture; 4 d | Hemp Node | | 5X IBA | putative | |
| | | | | | 16/8 | | | | epidermal | |
| | | | | | photoperiod | | | | | |
| WP1612- | Old | WCIC- | Badger | 1 min 100% | Rinsed 1X | 5 weeks | Rooted | 10 mg/L | | |
| 2a | | A-862 | | EtOH, then 5 | post | 50 mg/L | shoot | active | | |
| | | | | min 20% | inoculation; | active | | spectinomycin | | |
| | | | | Clorox; 37° C. | 2.25 ml | spectinomycin | | Hemp Rooting | | |
| | | | | imbibition in | co-culture; | Hemp Node | | minus carb | | |
| | | | | SDW | 4 d 16/8 | | | with 5X IBA | | |
| | | | | | photoperiod | | | | | |
| WP1331- | Hy- | DB22 | Badger | 1 min 100% | Rinsed 1X | 4 weeks | Rooted | 10 mg/L | Roots not | |
| 7a | brid | | | EtOH, then 5 | post | 75 mg/L | shoot | active | expressing | |
| | | | | min 20% | inoculation; | active | | spectinomycin | tdTOM | |
| | | | | Clorox; 37° C. | 2.25 ml | spectinomycin | | Hemp Rooting | but | |
| | | | | imbibition in | co-culture; | LIQUID | | minus carb | GUS+′ | |
| | | | | SDW | 4 d 16/8 | Hemp Node | | with 5X IBA | putative | |
| | | | | | photoperiod | | | | germline | |
| WP1331- | Old | DB22 | Badger | 5 min 20% | Rinsed 1X | 8 weeks | Rooted | Non- | Roots not | |
| 8a | | | | Clorox; 37° C. | post | 50 mg/L | shoot | selective | expressing | |
| | | | | imbibition in | inoculation; | active | | Hemp | tdTOM | |
| | | | | SDW | 2.25 ml | spectinomycin | | rooting with | but | |
| | | | | | co-culture; | Hemp Node | | 5X IBA | GUS+′ | |
| | | | | | 4 d 16/8 | with Glucose | | minus carb | putative | |
| | | | | | photoperiod | replacing | | | germline | |
| | | | | | | Sucrose | | | | |
| WP1331- | Hy- | DB22 | Badger | 1 min 100% | Rinsed 1X | 5 weeks | Rooted | 10 mg/L | tdTOM | |
| 9a | brid | | | EtOH, then 5 | post | 50 mg/L | shoot | active | positive | |
| | | | | min 20% | inoculation; | active | | spectinomycin | roots; | |
| | | | | Clorox; 37° C. | 2.25 ml co- | spectinomycin | | WPM | putative | |
| | | | | imbibition in | culture with | Hemp Node | | Hemp Rooting | germline; | |
| | | | | SDW | Sucrose | with Glucose | | minus carb | autoflowering | |
| | | | | | replacing | replacing | | with 5X IBA | | |
| | | | | | Glucose; | Sucrose | | | | |
| | | | | | 4 d 16/8 | | | | | |
| | | | | | photoperiod | | | | | |
| WP1845- | New | DB22 | Cherry | 5 min 20% | Primary | 4 weeks | Rooted | Non- | tdTOM | |
| 1a | | | Wine | Clorox; 37° C. | leaves | 50 mg/L | shoot | selective | positive | |
| | | | Hybrid | imbibition in | INTACT; | active | | WPM Hemp | roots; | |
| | | | | SDW | Rinsed 1X | spectinomycin | | Rooting | putative | |
| | | | | | post | LIQUID | | minus carb | germline | |
| | | | | | inoculation; | Hemp Node | | with 5X IBA | | |
| | | | | | 2.25 ml | | | | | |
| | | | | | co-culture; | | | | | |
| | | | | | 4 d 16/8 | | | | | |
| | | | | | photoperiod | | | | | |
| WP1344- | Old | WCIC- | Badger | 5 min 20% | 2.5 ml co- | 6 weeks | Rooted | 10 mg/L | | |
| 3a | | A-590 | | Clorox; 37° C. | culture; | 50 mg/L | shoot | active | | |
| | | | | imbibition in | 4 d 16/8 | active | | spectinomycin | | |
| | | | | SDW | photoperiod | spectinomycin | | with 5X | | |
| | | | | | | Hemp Node | | IBA minus | | |
| | | | | | | | | carb | | |
| WP1344- | Hy- | WCIC- | Badger | 5 min 20% | 1.75 ml | 9 weeks | Rooted | 10 mg/L | | |
| 4a, b | brid | A-590 | | Clorox; 37° C. | co-culture; | 50 mg/L | shoot | active | | |
| | | | | imbibition in | 3 d 16/8 | active | | spectinomycin | | |
| | | | | BGM | photoperiod | spectinomycin | | WPM | | |
| | | | | | | B5-based | | Hemp Rooting | | |
| | | | | | | Hemp Node | | minus carb | | |
| | | | | | | medium | | with 5X IBA | | |
| WP1508- | Hy- | WCIC- | Badger | 5 min 20% | Rinsed 1X | 4 weeks | Rooted | 10 mg/L | Roots | |
| 1a | brid | A-735 | | Clorox; 37° C. | post | 50 mg/L | shoot | active | aadA | |
| | | | | imbibition in | inoculation; | active | | spectinomycin | positive | |
| | | | | SDW | 2.25 ml | spectinomycin | | with 5X | by PCR; | |
| | | | | | co-culture; | LIQUID | | IBA minus | putative | |
| | | | | | 4 d 16/8 | Hemp Node | | carb | germline | |
| | | | | | photoperiod | | | | | |
| WP1508- | New | WCIC- | Badger | 1 min 100% | Primary | 5 weeks | Rooted | Non- | Roots | |
| 2a | | A-735 | | EtOH, then 5 | leaves | 50 mg/L | shoot | selective | aadA | |
| | | | | min 20% | INTACT; | active | | WPM Hemp | negative | |
| | | | | Clorox; 37° C. | Rinsed 1X | spectinomycin | | Rooting | by PCR; | |
| | | | | imbibition in | post | LIQUID | | minus carb | putative | |
| | | | | SDW | inoculation; | Hemp Node | | with 5X IBA | epidermal | |
| | | | | | 2.25 ml | | | | | |
| | | | | | co-culture; | | | | | |
| | | | | | 4 d 16/8 | | | | | |
| | | | | | photoperiod | | | | | |
| WP1851- | Hy- | LBA4404 | Badger | 5 min 20% | 2 ml co- | 1 week | Rooted | Non- | tdTOM | |
| 1a, b | brid | thy- + | | Clorox; 37° C. | culture | 50 mg/L | shoot | selective | positive | |
| | | H/DB22 | | imbibition in | supplemented | active | | WPM Hemp | roots; | |
| | | | | SDW | with 50 mg/L | spectinomycin | | Rooting | putative | |
| | | | | | thymidine; | Hemp Node; | | minus carb | germline; | |
| | | | | | 4 d 16/8 | then 2 weeks | | with 5X IBA | autoflowering | |
| | | | | | photoperiod | 50 mg/L | | | | |
| | | | | | | active | | | | |
| | | | | | | spectinomycin | | | | |
| | | | | | | Hemp Node | | | | |
| | | | | | | without | | | | |
| | | | | | | antibiotics | | | | |
| WP1331- | Old | DB22 | Badger | 5 min 20% | Rinsed 1X | 9 weeks | Rooted | Non- | tdTOM | |
| 10a | | | | Clorox; 37° C. | post | 50 mg/L | shoot | selective | positive | |
| | | | | imbibition in | inoculation; | active | | Hemp | roots; | |
| | | | | SDW | 2.25 ml | spectinomycin | | Rooting | putative | |
| | | | | | co-culture; | Hemp Node | | minus carb | germline | |
| | | | | | 4 d 16/8 | | | with 5X IBA | | |
| | | | | | photoperiod | | | | | |
| WP1507- | Old | WCIC- | Badger | 5 min 20% | Rinsed 1X | 6 weeks | Rooted | Non- | Roots | |
| 4a, b | | A-734 | | Clorox; 37° C. | post | 50 mg/L | shoot | selective | aadA | |
| | | | | imbibition in | inoculation; | active | | Hemp Rooting | positive | |
| | | | | SDW | 2 ml co- | spectinomycin | | minus carb | by PCR; | |
| | | | | | culture; 4 d | Hemp Node | | with 5X IBA | putative | |
| | | | | | 16/8 | | | | germline; -b | |
| | | | | | photoperiod | | | | plant | |
| | | | | | | | | | autoflowering | |
| WP1612- | Old | WCIC- | Badger | 5 min 20% | Rinsed 1X | 5 weeks 50 | Rooted | 10 mg/L | Roots | |
| 3a | | A-862 | | Clorox; 37° C. | post | mg/L | shoot | active | aadA | |
| | | | | imbibition in | inoculation; | active | | spectinomycin | positive | |
| | | | | SDW | 2.25 ml | spectinomycin | | Hemp Rooting | by PCR; | |
| | | | | | co-culture; | Hemp Node | | minus carb | putative | |
| | | | | | 4 d 16/8 | | | with 5X | germline | |
| | | | | | photoperiod | | | IBA; shoot | | |
| | | | | | | | | dipped in | | |
| | | | | | | | | 1000 mg/L | | |
| | | | | | | | | IBA | | |
| WP1344- | Hy- | WCIC- | Badger | 5 min 20% | 2.25 ml | 12 weeks | Rooted | 10 mg/L | | |
| 5a, b | brid | A-590 | | Clorox; 37° C. | co-culture; | 50 mg/L | shoot | active | | |
| | | | | imbibition in | 4 d 16/8 | active | | spectinomycin | | |
| | | | | SDW | photoperiod | spectinomycin | | WPM with | | |
| | | | | | | Hemp Node | | 5X IBA | | |
| | | | | | | without mT | | minus carb | | |
| WP1507- | Hy- | WCIC- | Badger | 5 min 20% | Rinsed 1X | 4 weeks | Rooted | Non- | Roots | |
| 5a, b | brid | A-734 | | Clorox; 37° C. | post | 50 mg/L | shoot | selective | aadA | |
| | | | | imbibition in | inoculation; | active | | Hemp WPM | positive | |
| | | | | SDW | 2 ml co- | spectinomycin | | Rooting | by PCR; | |
| | | | | | culture; 4 d | Hemp Node | | minus carb | putative | |
| | | | | | 16/8 | | | with 5X IBA | germline | |
| | | | | | photoperiod | | | | | |
| WP1331- | New | DB22 | Badger | 5 min 20% | Rinsed 1X | 4 weeks | Rooted | Non- | Roots not | |
| 11a | | | | Clorox; 37° C. | post | LIQUID | shoot | selective | expressing | |
| | | | | imbibition in | inoculation; | 50 mg/L | | Hemp WPM | RFP but | |
| | | | | SDW | 2.25 ml | active | | Rooting | expressing | |
| | | | | | co-culture; | spectinomycin | | minus carb | GUS; | |
| | | | | | 4 d 16/8 | Hemp Node | | with 5X IBA | putative | |
| | | | | | photoperiod | | | | germline | |
| WP1508- | New | WCIC- | Badger | 1 min 100% | Primary | 6 weeks | Rooted | 10 mg/L | | |
| 3a | | A-735 | | EtOH, then 5 | leaves | LIQUID | shoot | active | | |
| | | | | min 20% | INTACT; | 15 and | | spectinomycin | | |
| | | | | Clorox; 37° C. | Rinsed 1X | 50 mg/L | | WPM | | |
| | | | | imbibition in | post | active | | rooting with | | |
| | | | | SDW | inoculation; | spectinomycin | | 5X IBA | | |
| | | | | | 2.25 ml | Hemp Node | | minus carb | | |
| | | | | | co-culture; | | | | | |
| | | | | | 4 d 16/8 | | | | | |
| | | | | | photoperiod | | | | | |
| WP1612- | New | WCIC- | Badger | 5 min 20% | Primary | 4 weeks | Rooted | 10 mg/L | | |
| 4a | | A-862 | | Clorox; 37° C. | leaves | LIQUID | shoot | active | | |
| | | | | imbibition in | INTACT; | 50 mg/L | | spectinomycin | | |
| | | | | SDW | Rinsed 1X | active | | WPM | | |
| | | | | | post | spectinomycin | | rooting with | | |
| | | | | | inoculation; | Hemp Node | | 5X IBA | | |
| | | | | | 2.25 ml | | | minus carb | | |
| | | | | | co-culture; | | | | | |
| | | | | | 4 d 16/8 | | | | | |
| | | | | | photoperiod | | | | | |
| WP1853- | New | DICOT | Badger | 5 min 20% | Primary | 3.5 weeks | Rooted | 10 mg/L | | |
| 1a | | RUBYv1 | | Clorox; 37° C. | leaves | LIQUID | shoot | active | | |
| | | (WCIC- | | imbibition in | INTACT; | 50 mg/L | | spectinomycin | | |
| | | A-989) | | SDW | Rinsed 1X | active | | WPM | | |
| | | | | | post | spectinomycin | | rooting with | | |
| | | | | | inoculation; | Hemp Node | | 5X IBA | | |
| | | | | | 2.25 ml | | | minus carb | | |
| | | | | | co-culture; | | | | | |
| | | | | | 4 d 16/8 | | | | | |
| | | | | | photoperiod | | | | | |
| WP1851- | Hy- | LBA4404 | Badger | 5 min 20% | 2 ml co- | 1 week | Rooted | Non- | Roots not | |
| 2a, b | brid | thy- + | | Clorox; 37° C. | culture | 50 mg/L | shoot | selective | expressing | |
| | | H/DB22 | | imbibition in | supplemented | active | | Hemp WPM | RFP | |
| | | | | SDW | with 50 mg/L | spectinomycin | | Rooting | but | |
| | | | | | thymidine; | Hemp Node; | | minus carb | expressing | |
| | | | | | 4 d 16/8 | then 3 weeks | | with 5X IBA | GUS; | |
| | | | | | photoperiod | 50 mg/L | | | putative | |
| | | | | | | active | | | germline | |
| | | | | | | spectinomycin | | | | |
| | | | | | | Hemp Node | | | | |
| | | | | | | without | | | | |
| | | | | | | antibiotics | | | | |
| WP1507- | Hy- | WCIC- | Badger | 5 min 20% | Rinsed 1X | 7 weeks | Rooted | Non- | Roots | |
| 6a | brid | A-734 | | Clorox; 37° C. | post | 50 mg/L | shoot | selective | PCR | |
| | | | | imbibition in | inoculation; | active | | Hemp WPM | negative | |
| | | | | SDW | 2.25 ml | spectinomycin | | Rooting | for aadA; | |
| | | | | | co-culture; | Hemp Node | | minus carb | putative | |
| | | | | | 4 d 16/8 | | | with 5X IBA | epidermal | |
| | | | | | photoperiod | | | | | |
| WP1612- | New | WCIC- | Badger | 5 min 20% | Primary | 4.5 weeks | Rooted | 10 mg/L | Roots | |
| 5a | | A-862 | | Clorox; 37° C. | leaves | LIQUID | shoot | active | aadA+ | |
| | | | | imbibition in | INTACT; | 50 mg/L | | spectinomycin | by PCR; | |
| | | | | SDW | Rinsed 1X | active | | WPM | putative | |
| | | | | | post | spectinomycin | | rooting with | germline | |
| | | | | | inoculation; | Hemp Node | | 5X IBA | | |
| | | | | | 2.25 ml | with 1 mg/L | | minus carb | | |
| | | | | | co-culture; | GA3 | | | | |
| | | | | | 4 d 16/8 | | | | | |
| | | | | | photoperiod | | | | | |
| WP1331- | New | DB22 | Badger | 5 min 20% | Primary | 4 weeks | Rooted | 10 mg/L | Roots | |
| 12a | | | | Clorox; 37° C. | leaves | LIQUID | shoot | active | GUS+; | |
| | | | | imbibition in | INTACT; | 50 mg/L | | spectinomycin | putative | |
| | | | | SDW | Rinsed 1X | active | | WPM rooting | germline | |
| | | | | | post | spectinomycin | | with 5X IBA | | |
| | | | | | inoculation; | Hemp Node | | minus carb | | |
| | | | | | 2.25 ml | | | | | |
| | | | | | co-culture; | | | | | |
| | | | | | 4 d 16/8 | | | | | |
| | | | | | photoperiod | | | | | |
| WP1853- | New | DICOT | Badger | 5 min 20% | Primary | 3.5 weeks | Rooted | 10 mg/L | Roots | |
| 2a | | RUBYv1 | | Clorox; 37° C. | leaves | LIQUID | shoot | active | aadA+ | |
| | | (WCIC- | | imbibition in | INTACT; | 50 mg/L | | spectinomycin | by PCR; | |
| | | A-989) | | SDW | Rinsed 1X | active | | WPM rooting | putative | |
| | | | | | post | spectinomycin | | with 5X IBA | germline | |
| | | | | | inoculation; | Hemp Node | | minus carb | | |
| | | | | | 2.25 ml | | | | | |
| | | | | | co-culture; | | | | | |
| | | | | | 4 d 16/8 | | | | | |
| | | | | | photoperiod | | | | | |
| WP1853- | New | DICOT | Badger | 5 min 20% | Primary | 3.5 weeks | Rooted | 10 mg/L | Roots | |
| 3a | | RUBYv1 | | Clorox; 37° C. | leaves | LIQUID | shoot | active | aadA+ | |
| | | (WCIC- | | imbibition in | INTACT; | 50 mg/L | | spectinomycin | by PCR; | |
| | | A-989) | | SDW | Rinsed 1X | active | | WPM rooting | putative | |
| | | | | | post | spectinomycin | | with 5X IBA | germline | |
| | | | | | inoculation; | Hemp Node | | minus carb | | |
| | | | | | 2.25 ml | | | | | |
| | | | | | co-culture; | | | | | |
| | | | | | 4 d 16/8 | | | | | |
| | | | | | photoperiod | | | | | |
| WP1853- | New | DICOT | Badger | 5 min 20% | Primary | 3.5 weeks | Rooted | 10 mg/L | | |
| 4a | | RUBYv1 | | Clorox; 37° C. | leaves | LIQUID | shoot | active | | |
| | | (WCIC- | | imbibition in | INTACT; | 50 mg/L | | spectinomycin | | |
| | | A-989) | | SDW | Rinsed 1X | active | | WPM rooting | | |
| | | | | | post | spectinomycin | | with 5X IBA | | |
| | | | | | inoculation; | Hemp Node | | minus carb | | |
| | | | | | 2.25 ml | | | | | |
| | | | | | co-culture; | | | | | |
| | | | | | 4 d 16/8 | | | | | |
| | | | | | photoperiod | | | | | |
| WP1853- | New | DICOT | Badger | 5 min 20% | Primary | 4.5 weeks | Rooted | 10 mg/L | Roots | |
| 5a | | RUBYv1 | | Clorox; 37° C. | leaves | LIQUID | shoot | active | expressing | |
| | | (WCIC- | | imbibition in | INTACT; | 50 mg/L | | spectinomycin | DICOT | |
| | | A-989) | | SDW | Rinsed 1X | active | | WPM rooting | RUBYv1; | |
| | | | | | post | spectinomycin | | with 5X IBA | putative | |
| | | | | | inoculation; | Hemp Node | | minus carb | germline | |
| | | | | | 2.25 ml | | | | | |
| | | | | | co-culture; | | | | | |
| | | | | | 4 d 16/8 | | | | | |
| | | | | | photoperiod | | | | | |
| WP1508- | New | WCIC- | Badger | 1 min 100% | Primary | 8 weeks | Rooted | 10 mg/L | Splotchy, | |
| 4a | | A-735 | | EtOH, then 5 | leaves | LIQUID | shoot | active | likely | |
| | | | | min 20% | INTACT; | 15 and | | spectinomycin | chimeric | |
| | | | | Clorox; 37° C. | Rinsed 1X | 50 mg/L | | WPM rooting | | |
| | | | | imbibition in | post | active | | with 5X IBA | | |
| | | | | SDW | inoculation; | spectinomycin | | minus carb | | |
| | | | | | 2.25 ml | Hemp Node | | | | |
| | | | | | co-culture; | | | | | |
| | | | | | 4 d 16/8 | | | | | |
| | | | | | photoperiod | | | | | |
| WP1508- | New | WCIC- | Badger | 5 min 20% | Primary | 8 weeks | Rooted | 10 mg/L | Splotchy, | |
| 5a | | A-735 | | Clorox; 37° C. | leaves | LIQUID | shoot | active | likely | |
| | | | | imbibition in | INTACT; | 15 and | | spectinomycin | chimeric | |
| | | | | SDW | Rinsed 1X | 50 mg/L | | WPM rooting | | |
| | | | | | post | active | | with 5X IBA | | |
| | | | | | inoculation; | spectinomycin | | minus carb | | |
| | | | | | 2.25 ml | Hemp Node | | | | |
| | | | | | co-culture; | | | | | |
| | | | | | 4 d 16/8 | | | | | |
| | | | | | photoperiod | | | | | |
| WP1508- | New | WCIC- | Badger | 5 min 20% | Primary | 8 weeks | Rooted | 10 mg/L | | |
| 6a | | A-735 | | Clorox; 37° C. | leaves | LIQUID 15 | shoot | active | | |
| | | | | imbibition in | INTACT; | and 50 | | spectinomyci | | |
| | | | | SDW | Rinsed 1X | mg/L | | n WPM | | |
| | | | | | post | active | | rooting with | | |
| | | | | | inoculation; | spectinomycin | | 5X IBA | | |
| | | | | | 2.25 ml | Hemp Node | | minus carb | | |
| | | | | | co-culture; | | | | | |
| | | | | | 4 d 16/8 | | | | | |
| | | | | | photoperiod | | | | | |
| WP1612- | New | WCIC- | Badger | 5 min 20% | Primary | 8.5 weeks | Rooted | 10 mg/L | | |
| 6a | | A-862 | | Clorox; 37° C. | leaves | LIQUID | shoot | active | | |
| | | | | | INTACT; | 15 and | | spectinomycin | | |
| | | | | | Rinsed 1X | 50 mg/L | | WPM rooting | | |
| | | | | | post | active | | with 5X IBA | | |
| | | | | | inoculation; | spectinomycin | | minus carb | | |
| | | | | | 2.25 ml | Hemp Node | | | | |
| | | | | | co-culture; | | | | | |
| | | | | | 4 d 16/8 | | | | | |
| | | | | | photoperiod | | | | | |
| WP1612- | New | WCIC- | Badger | 5 min 20% | Primary | 8.5 weeks | Rooted | 10 mg/L | | |
| 7a | | A-862 | | Clorox; 37° C. | leaves | LIQUID | shoots | active | | |
| | | | | | INTACT; | 15 and | | spectinomycin | | |
| | | | | | Rinsed 1X | 50 mg/L | | WPM rooting | | |
| | | | | | post | active | | with 5X IBA | | |
| | | | | | inoculation; | spectinomycin | | minus carb | | |
| | | | | | 2.25 ml | Hemp Node + | | | | |
| | | | | | co-culture; | 1 ppm GA3 | | | | |
| | | | | | 4 d 16/8 | | | | | |
| | | | | | photoperiod | | | | | |
| WP1331- | New | DB22 | Badger | 5 min 20% | Primary | 3.5 weeks | Rooted | 10 mg/L | Roots | |
| 13a | | | | Clorox; 37° C. | leaves | LIQUID | shoot | active | RFP+, | |
| | | | | | INTACT; | 15 and | | spectinomycin | putative | |
| | | | | | Rinsed 1X | 50 mg/L | | WPM rooting | germline | |
| | | | | | post | active | | with 5X IBA | | |
| | | | | | inoculation; | spectinomycin | | minus carb | | |
| | | | | | 2.25 ml | Hemp Node | | | | |
| | | | | | co-culture; | | | | | |
| | | | | | 4 d dark | | | | | |

### Example 3

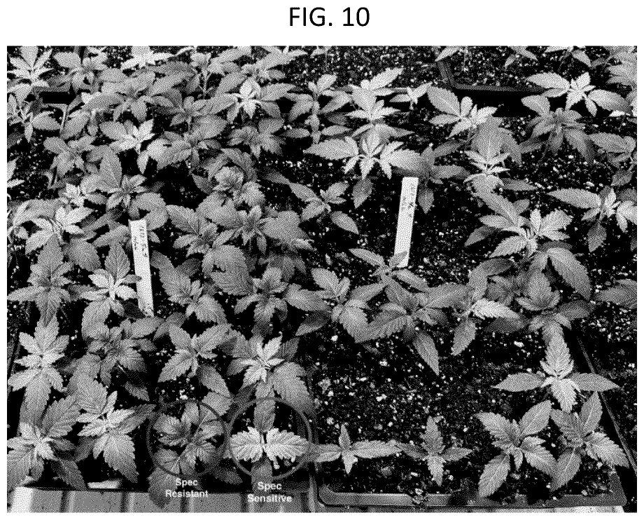

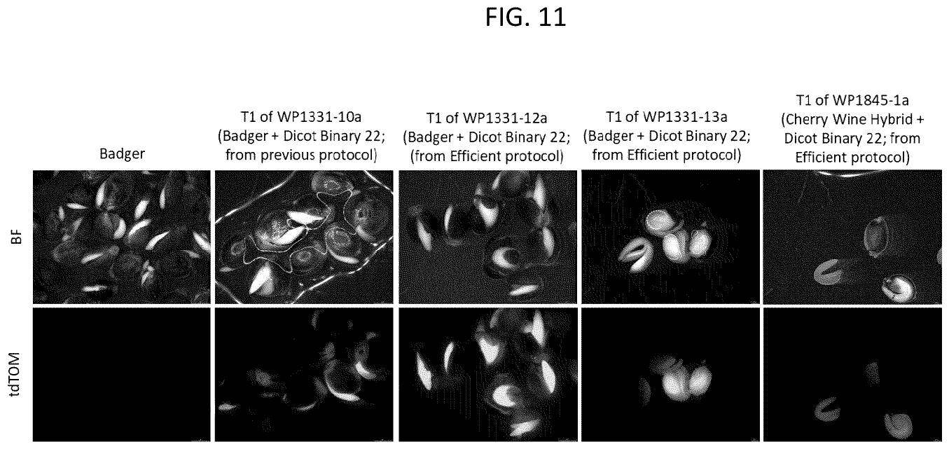

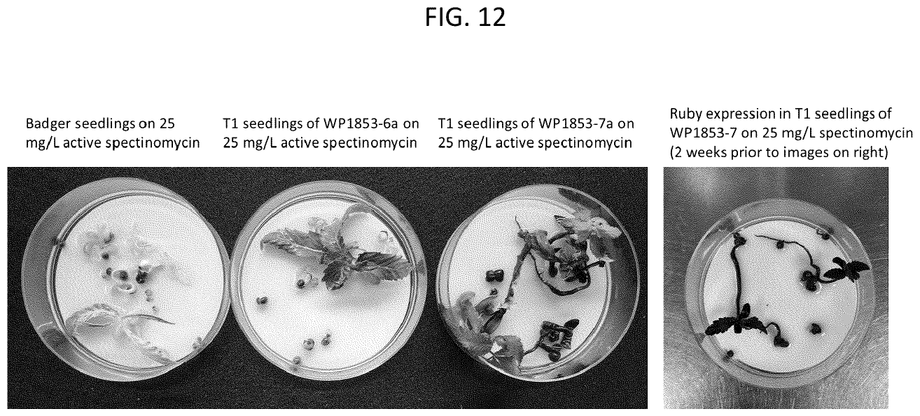

While previously, germline rates (T1) were predicted from TO Cannabis shoots rooting on selection and/or presence of transgene in TO root tissue, the following example illustrates the generation of stable T1 Cannabis plants. Transformation frequencies and rates for T1 plants are reported in Table 2. T1 Germline status was established with the WCIC-A-862 construct by observing greening/bleaching of greenhouse-grown seedlings sprayed with 1000 mg/L spectinomycin (FIG. 10). T1 Germline status was established with the DICOTBINARY22 control construct by observing tdTOM presence in the T1 embryo (FIG. 11). T1 Germline status was established with the WCIC-A-989 DICOT RUBYv1 control construct by observing betanin presence in the developing plant, or by looking for spectinomycin resistance in developing seedlings (FIG. 12).

### Efficient Cannabis Transformation Using Gaantry System:

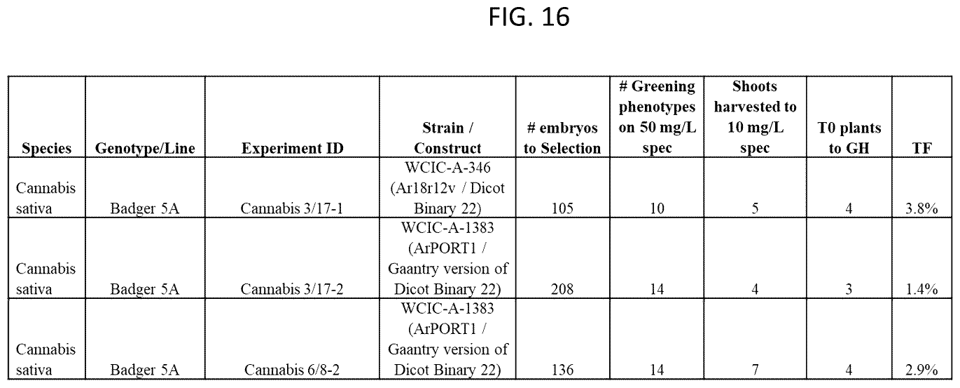

The GAANTRY (Gene Assembly in Agrobacterium by Nucleic acid sTacking using Recombinase technologY) system may also be used for transformation of explants. We ran proof of concept experiments in Cannabis meristems using T-DNA launched from the disarmed virulence/Ri plasmid (Collier 2018) rather than T-DNA launched from a binary plasmid and were able to recover T0 plants from this GAANTRY (Gene Assembly in Agrobacterium by Nucleic acid sTacking using Recombinase technologY) system (FIG. 14). The first TO plants from GAANTRY rooted in the presence of spectinomycin, with one plant expressing tdTOM and the other expressing GUS, are shown in FIG. 15. Transformation metrics from conventional binary strategy compared to Gaantry are given in FIG. 16.

### Example 4

Additional modifications to the protocols described in Examples 1-3 are described below.

### Protocol Modifications:

Alternate media schedules. We examined feeding explants a lower volume of liquid media at greater frequency than our standard treatment, but this did not appear advantageous save for offering greater flexibility to the feeding schedule (Table 11).

TABLE 11
Alternative Media Schedule Experiments

| | | | | | | # Greening | Shoots | | |
|---|---|---|---|---|---|---|---|---|---|
| | | | | # embryos | | phenotypes | harvested | T0 | |
| | Genotype/ | Experiment | Strain/ | to | | on 50 | to 10 | plants | |
| Species | Line | ID | Construct | Selection | Transfer regime | mg/L spec | mg/L spec | to GH | TF |
| Cannabis | Badger | Cannabis | WCIC-A-346 | 70 | 2.3 ml co-culture | 3 | 1 | 1 | 1.4% |
| sativa | 5A | 7/14-1 | (Ar18r12v/ | | volume, 15 ml initial | | | | |
| | | | DB22) | | liquid media, 10 ml | | | | |
| | | | | | subsequent feedings | | | | |
| | | | | | every ~10 days | | | | |
| Cannabis | Badger | Cannabis | WCIC-A-346 | 80 | 2.5 ml co-culture | 4 | 3 | 2 | 2.5% |
| sativa | 5A | 7/14-2 | (Ar18r12v/ | | volume, 15 ml initial | | | | |
| | | | DB22) | | liquid media, 10 ml | | | | |
| | | | | | subsequent feedings | | | | |
| | | | | | every ~10 days | | | | |
| Cannabis | Badger | Cannabis | WCIC-A-346 | 80 | 2.3 ml co-culture | 1 | 1 | 1 | 1.3% |
| sativa | 5A | 7/14-3 | (Ar18r12v/ | | volume, 15 ml initial | | | | |
| | | | DB22) | | liquid media, 5 ml | | | | |
| | | | | | subsequent feedings | | | | |
| | | | | | every ~7 days | | | | |
| Cannabis | Badger | Cannabis | WCIC-A-346 | 80 | 2.5 ml co-culture | 2 | 2 | 0 | 0.0% |
| sativa | 5A | 7/14-4 | (Ar18r12v/ | | volume, 15 ml initial | | | | |
| | | | DB22) | | liquid media, 5 ml | | | | |
| | | | | | subsequent feedings | | | | |
| | | | | | every ~7 days | | | | |

Modified media. We also examined alternate medias during the selection/regeneration phase, as described in Table 12 below. The first set of these experiments examined varying levels of ammonium nitrate and potassium nitrate in the media. We found lowering the ammonium nitrate concentration did not appear advantageous over the standard (although in this set the standard treatment did not produce TO plants). We did obtain a T0 plant by increasing the ammonium nitrate concentration from the std MS level (1650 mg/L) to 2500 mg/L. We also obtained plants from DKW, which has a comparable level of ammonium nitrate but a lower amount of potassium nitrate than MS media. For example, we obtained plants utilizing a modified DKW, which contains 950 mg/L potassium nitrate compared to the standard 0 mg/L potassium nitrate.

TABLE 12
Modified Media Experiments

| | | | | | Transfer | | | # Greening | Shoots | | |
|---|---|---|---|---|---|---|---|---|---|---|---|
| | | | | # embryos | regime | | | phenotypes | harvested | T0 | |
| | Genotype/ | Experiment | Strain/ | to | (with 0.5 | Ammonium | Potassium | on 50 | to 10 | plants | |
| Species | Line | ID | Construct | Selection | mg/L mT) | nitrate | nitrate | mg/L spec | mg/L spec | to GH | TF |
| Cannabis | Badger | Cannabis | WCIC-A-346 | 96 | Hemp node | 0 | 1900 | 2 | 0 | 0 | 0.0% |
| sativa | 5A | 9/14-1 | (Ar18r12v/ | | media (MS- | mg/L | mg/L | | | | |
| | | | DB22) | | based) | | | | | | |
| | | | | | without | | | | | | |
| | | | | | ammonium | | | | | | |
| | | | | | nitrate | | | | | | |
| Cannabis | Badger | Cannabis | WCIC-A-346 | 96 | Hemp node | 400 | 1900 | 6 | 0 | 0 | 0.0% |
| sativa | 5A | 9/14-2 | (Ar18r12v/ | | media (MS- | mg/L | mg/L | | | | |
| | | | DB22) | | based) | | | | | | |
| | | | | | with | | | | | | |
| | | | | | 400 mg/L | | | | | | |
| | | | | | ammonium | | | | | | |
| | | | | | nitrate | | | | | | |
| Cannabis | Badger | Cannabis | WCIC-A-346 | 96 | Hemp node | 800 | 1900 | 2 | 2 | 0 | 0.0% |
| sativa | 5A | 9/14-3 | (Ar18r12v/ | | media (MS- | mg/L | mg/L | | | | |
| | | | DB22) | | based) | | | | | | |
| | | | | | with | | | | | | |
| | | | | | 800 mg/L | | | | | | |
| | | | | | ammonium | | | | | | |
| | | | | | nitrate | | | | | | |
| Cannabis | Badger | Cannabis | WCIC-A-346 | 96 | Hemp node | 1650 | 1900 | 1 | 0 | 0 | 0.0% |
| sativa | 5A | 9/14-4 | (Ar18r12v/ | | media (MS- | mg/L | mg/L | | | | |
| | | | DB22) | | based) | | | | | | |
| | | | | | with | | | | | | |
| | | | | | 1650 mg/L | | | | | | |
| | | | | | ammonium | | | | | | |
| | | | | | nitrate | | | | | | |
| | | | | | (STD) | | | | | | |
| Cannabis | Badger | Cannabis | WCIC-A-346 | 48 | Hemp node | 2500 | 1900 | 1 | 1 | 1 | 2.1% |
| sativa | 5A | 9/14-5 | (Ar18r12v/ | | media (MS- | mg/L | mg/L | | | | |
| | | | DB22) | | based) | | | | | | |
| | | | | | with | | | | | | |
| | | | | | 2500 mg/L | | | | | | |
| | | | | | ammonium | | | | | | |
| | | | | | nitrate | | | | | | |
| Cannabis | Badger | Cannabis | WCIC-A-346 | 96 | Hemp node | 1650 | 1900 | 1 | 1 | 0 | 0.0% |
| sativa | 5A | 9/29-1 | (Ar18r12v/ | | media (MS- | mg/L | mg/L | | | | |
| | | | DB22) | | based) | | | | | | |
| | | | | | with | | | | | | |
| | | | | | 1650 mg/L | | | | | | |
| | | | | | ammonium | | | | | | |
| | | | | | nitrate | | | | | | |
| | | | | | (STD) | | | | | | |
| Cannabis | Badger | Cannabis | WCIC-A-346 | 96 | DKW | 1416 | 0 | 5 | 4 | 1 | 1.0% |
| sativa | 5A | 9/29-2 | (Ar18r12v/ | | media | mg/L | mg/L | | | | |
| | | | DB22) | | | | | | | | |
| Cannabis | Badger | Cannabis | WCIC-A-346 | 96 | modified | 1416 | 950 | 12 | 6 | 3 | 3.1% |
| sativa | 5A | 9/29-3 | (Ar18r12v/ | | DKW | mg/L | mg/L | | | | |
| | | | DB22) | | media | | | | | | |
| Cannabis | Badger | Cannabis | WCIC-A-346 | 96 | WPM | 400 | 0 | 0 | 0 | 0 | 0.0% |
| sativa | 5A | 9/29-4 | (Ar18r12v/ | | media | mg/L | mg/L | | | | |
| | | | DB22) | | | | | | | | |

We also examined the impact of replacing meta-topolin with Phytoax cytokinin in the modified cytokinin experiments, as shown in Table 13 below. Phytoax did not appear advantageous over meta-topolin, but again obtained T0 plants using DKW media as an alternative to MS media.

TABLE 13
Meta-topolin v. Phytoax in Media

| | | | | | | # Greening | Shoots | | |
|---|---|---|---|---|---|---|---|---|---|
| | | | | # embryos | | phenotypes | harvested | T0 | |
| | Genotype/ | | | to | | on 50 | to 10 | plants | |
| Species | Line | Co-culture | Experiment | Selection | Transfer regime | mg/L spec | mg/L spec | to GH | TF |
| Cannabis | Badger 5D | 2.25 ml INO + 1 | Cannabis | 112 | Hemp node | 5 | 3 | 1 | 0.9% |
| sativa | | ppm TDZ + | 10/6-1 | | media (MS- | | | | |
| | | nys/TBZ; 23 C. | | | based) with 1650 | | | | |
| | | 16/8 | | | mg/L ammonium | | | | |
| | | photoperiod | | | nitrate with 0.5 | | | | |
| | | | | | mg/L mT (STD) | | | | |
| Cannabis | Badger 5D | 2.25 ml INO + 1 | Cannabis | 112 | Hemp node | 8 | 3 | 1 | 0.9% |
| sativa | | ppm TDZ + | 10/6-2 | | media (MS- | | | | |
| | | nys/TBZ; 23 C. | | | based) with 1650 | | | | |
| | | 16/8 | | | mg/L ammonium | | | | |
| | | photoperiod | | | nitrate with 0.5 | | | | |
| | | | | | mg/L Phytoax | | | | |
| Cannabis | Badger 5D | 2.25 ml INO + 1 | Cannabis | 112 | DKW media with | 7 | 2 | 2 | 1.8% |
| sativa | | ppm TDZ + | 10/6-3 | | 0.5 mg/L mT | | | | |
| | | nys/TBZ; 23 C. | | | | | | | |
| | | 16/8 | | | | | | | |
| | | photoperiod | | | | | | | |
| Cannabis | Badger 5D | 2.25 ml INO + 1 | Cannabis | 112 | DKW media with | 2 | 0 | 0 | 0.0% |
| sativa | | ppm TDZ + | 10/6-4 | | 0.5 mg/L Phytoax | | | | |
| | | nys/TBZ; 23 C. | | | | | | | |
| | | 16/8 | | | | | | | |
| | | photoperiod | | | | | | | |

### Example 5

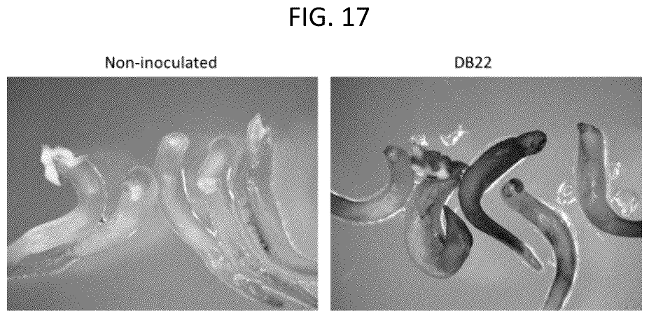

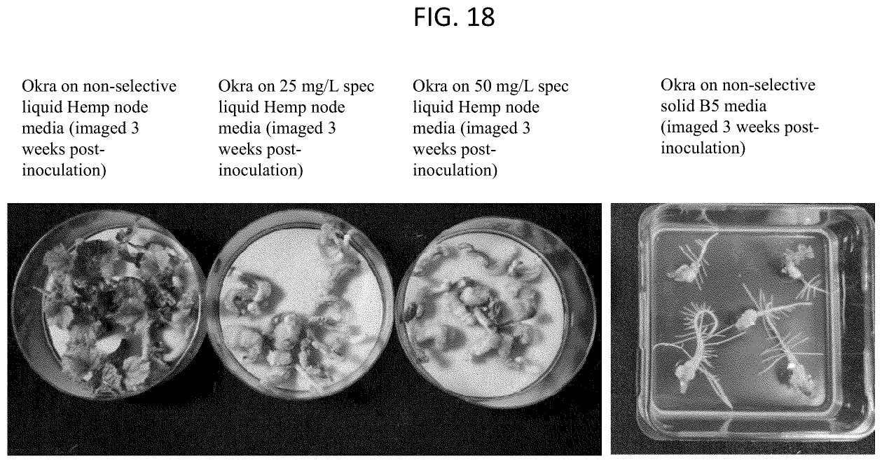

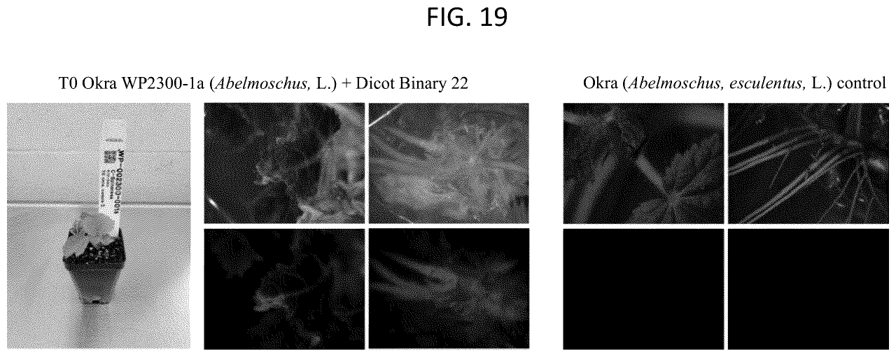

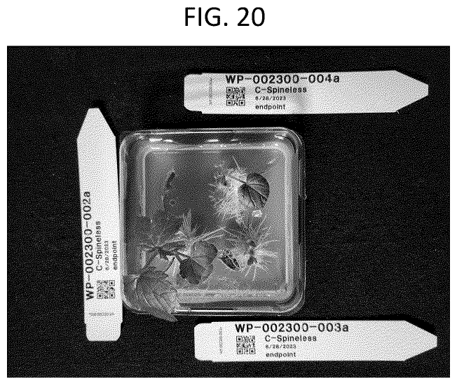

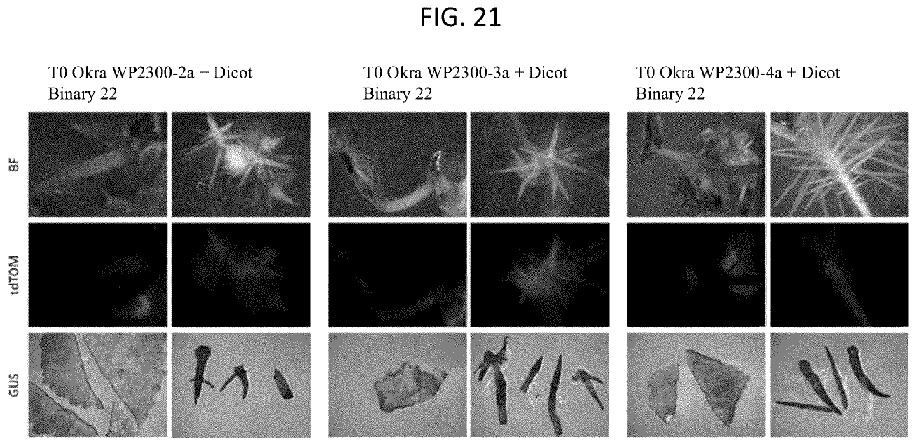

The following example describes efficient transformation in Okra (Abelmoschus esculentus, L.). We successively obtained germline TO Okra transgenic plants through the Efficient Cannabis Transformation process. For Okra transformation we used a greater co-culture volume (2.5 ml/plantcon) then we generally use for Cannabis as Okra meristem explants physically larger than Cannabis meristem explants (although we have generated germline events from this volume in Cannabis as well). We imaged transient GUS expression in Okra meristem explants post co-culture, as shown in FIG. 17 (right panel), compared to a non-inoculated control (left panel). Okra explant phenotypes are shown in FIG. 18 on non-selective MS liquid media (far left image of left panel) and on a solid B5 media (right panel), along with inoculated explants on 25-50 mg/L spectinomycin selection (middle and right images in left panel). Stable tdTOM expression in T0 Okra roots of the first plant 7.5 weeks post-inoculation is shown in FIG. 19. Explants followed the “Efficient Cannabis meristem tfn protocol”. TO Okra phenotypes from plants generated with the “Efficient Cannabis transformation” process are shown in FIG. 20. Stable tdTOM (roots) and GUS expression (roots, leaves) in T0 Okra plants derived from “Efficient Cannabis transformation” process are shown in FIG. 21. Images captured 8.5 weeks post-inoculation. Explants followed “Efficient Cannabis meristem tfn protocol” with 25 mg/L active spectinomycin during regeneration/selection.

In the pilot Okra meristem transformation tests, we used hand excised meristems (primary leaves intact) from seed surface sanitized in 20% Clorox 5 min; rinsed; imbibed for ˜20 h in H2O at 37 C; rinsed, inoculated and sonicated 20s 45 kHz; incubated 30 min, inoculum removed; explants co-cultured on 2.5 ml INO+1 ppm TDZ+nys/TBZ; 23 C 16/8 photoperiod. Transformation metrics in Pilot Okra meristem transformation test are shown in Table 14 below.

TABLE 14
Transformation metrics in Pilot Okra meristem transformation test

| | | | | | | | | # Germline | |
|---|---|---|---|---|---|---|---|---|---|
| | | Experiment | # embryos | First selection | # | #T0 | Putative | Plants (T1 | Germline |
| Species | Genotype/Line | ID | to Selection | media | Shoots | plants | TF | pos seed) | TF |
| Okra | Clemson | Okra 4/27-1 | 5 | Non-selective | n/a | n/a | n/a | n/a | n/a |
| | Spineless OG | | | B5 (solid) | | | | | |
| Okra | Clemson | Okra 4/27-2 | 25 | 25 mg/L | 1 | 0 | 0.0% | 0 | 0.0% |
| | Spineless OG | | | spectinomycin | | | | | |
| | | | | B5 (solid) | | | | | |
| Okra | Clemson | Okra 4/27-3 | 25 | 150 mg/L | 1 | 1 | 4.0% | 1 | 4.0% |
| | Spineless OG | | | spectinomycin | | | | | |
| | | | | B5 (solid) | | | | | |
| Okra | Clemson | Okra 4/27-4 | 7 | Non-selective | n/a | n/a | n/a | n/a | n/a |
| | Spineless OG | | | hemp node | | | | | |
| | | | | with 0.5 ppm | | | | | |
| | | | | mT (liquid) | | | | | |
| Okra | Clemson | Okra 4/27-5 | 20 | 25 mg/L | 4 | 3 | 15.0% | 3 | 15.0% |
| | Spineless OG | | | spectinomycin | | | | | |
| | | | | hemp node | | | | | |
| | | | | with 0.5 ppm | | | | | |
| | | | | mT (liquid) | | | | | |
| Okra | Clemson | Okra 4/27-6 | 20 | 50 mg/L | 5 | 2 | 10.0% | 0 (1 plant | n/a |
| | Spineless OG | | | spectinomycin | | | | tossed | |
| | | | | hemp node | | | | before seed) | |
| | | | | with 0.5 ppm | | | | | |
| | | | | mT (liquid) | | | | | |

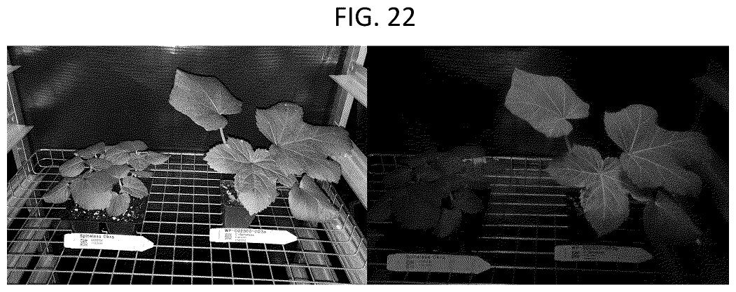

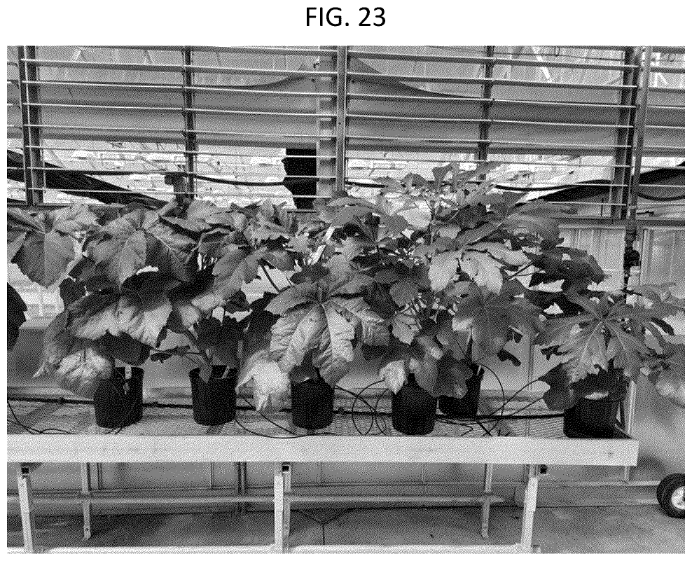

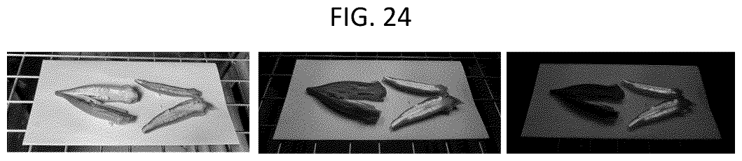

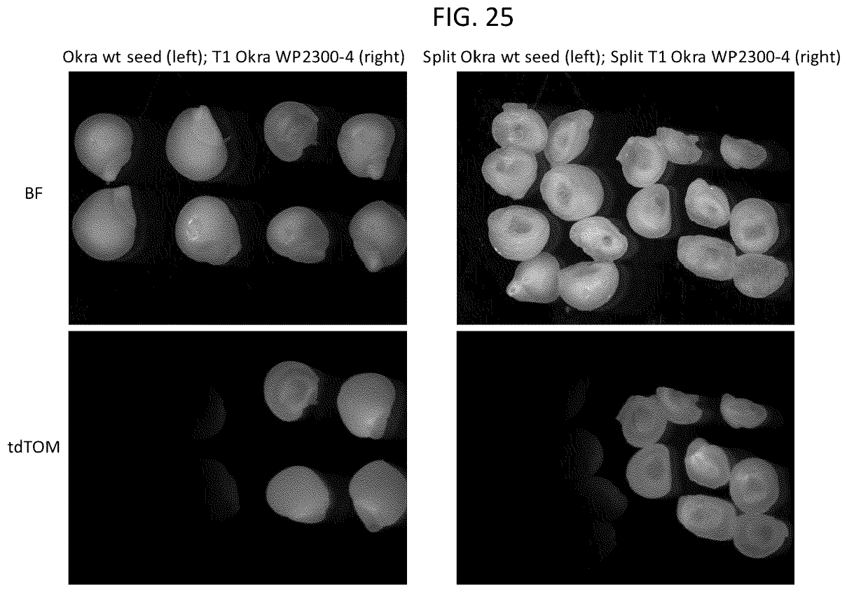

FIG. 22 shows stable tdTOM expression in TO Okra event WP2300-3a (right plant in both panels) ˜1 month after handoff; the control plant is on the left in both panels (FIG. 22). FIG. 23 shows examples of T0 Okra plant phenotypes in the greenhouse. FIG. 24 shows Okra conventional pod/seed vs. tdTOM expression in T1 Okra pod/seed of WP2300-4a). FIG. 25 shows Okra conventional seeds (top left panel) and conventional split seeds (top right panel) vs. tdTOM expression in T1 Okra seeds (bottom left panel) and tdTOM expression in T1 Okra split seeds (bottom right panel) of WP2300-4a.

Transformation metrics from a follow-up experiment of Okra transformation (TO plants not sent to GH) are shown in Table 15 below.

TABLE 15
Transformation Metrics of Okra Using Solid v. Liquid Media and Different Rates of Spectinomycin Selection

| | | | | # embryos | | | | |
|---|---|---|---|---|---|---|---|---|
| | | | Experiment | to | First selection | # | #T0 | Putative |
| Species | Genotype | Comments | ID | Selection | media | Shoots | plants | TF |
| Okra | Clemson | Hand excised (primary leaves | Okra 6/1-1 | 50 | 25 mg/L | 2 | 2 | 4.0% |
| | Spineless | intact) from seed surface | | | spectinomycin | | | |
| | OG | sanitized in 20% Clorox 5 | | | Hemp node | | | |
| | | min; rinsed; imbibed for ~20 | | | with 0.5 ppm | | | |
| | | h in H2O at 37 C.; rinsed, | | | mT (solid) | | | |
| | | inoculated and sonicated 2 | | | | | | |
| | | min 45 kHz; incubated 30 | | | | | | |
| | | min, inoculum removed; | | | | | | |
| | | explants co-cultured on 2.5 | | | | | | |
| | | ml INO + 1 ppm TDZ + | | | | | | |
| | | nys/TBZ; 23 C. 16/8 | | | | | | |
| | | photoperiod | | | | | | |
| Okra | Clemson | Hand excised (primary leaves | Okra 6/1-2 | 50 | 50 mg/L | 7 | 6 | 12.0% |
| | Spineless | intact) from seed surface | | | spectinomycin | | | |
| | OG | sanitized in 20% Clorox 5 | | | Hemp node | | | |
| | | min; rinsed; imbibed for ~20 | | | with 0.5 ppm | | | |
| | | h in H2O at 37 C.; rinsed, | | | mT (solid) | | | |
| | | inoculated and sonicated 2 | | | | | | |
| | | min 45 kHz; incubated 30 | | | | | | |
| | | min, inoculum removed; | | | | | | |
| | | explants co-cultured on 2.5 | | | | | | |
| | | ml INO + 1 ppm TDZ + | | | | | | |
| | | nys/TBZ; 23 C. 16/8 | | | | | | |
| | | photoperiod | | | | | | |
| Okra | Clemson | Hand excised (primary leaves | Okra 6/1-3 | 48 | 25 mg/L | 20 | 8 | 16.7% |
| | Spineless | intact) from seed surface | | | spectinomycin | | | |
| | OG | sanitized in 20% Clorox 5 | | | Hemp node | | | |
| | | min; rinsed; imbibed for ~20 | | | with 0.5 ppm | | | |
| | | h in H2O at 37 C.; rinsed, | | | mT (15 ml | | | |
| | | inoculated and sonicated 2 | | | liquid on 4 | | | |
| | | min 45 kHz; incubated 30 | | | filter papers) | | | |
| | | min, inoculum removed; | | | | | | |
| | | explants co-cultured on 2.5 | | | | | | |
| | | ml INO + 1 ppm TDZ + | | | | | | |
| | | nys/TBZ; 23 C. 16/8 | | | | | | |
| | | photoperiod | | | | | | |
| Okra | Clemson | Hand excised (primary leaves | Okra 6/1-4 | 51 | 50 mg/L | 16 | 9 | 17.6% |
| | Spineless | intact) from seed surface | | | spectinomycin | | | |
| | OG | sanitized in 20% Clorox 5 | | | Hemp node | | | |
| | | min; rinsed; imbibed for ~20 | | | with 0.5 ppm | | | |
| | | h in H2O at 37 C.; rinsed, | | | mT (15 ml | | | |
| | | inoculated and sonicated 2 | | | liquid on 4 | | | |
| | | min 45 kHz; incubated 30 | | | filter papers) | | | |
| | | min, inoculum removed; | | | | | | |
| | | explants co-cultured on 2.5 | | | | | | |
| | | ml INO + 1 ppm TDZ + | | | | | | |
| | | nys/TBZ; 23 C. 16/8 | | | | | | |
| | | photoperiod | | | | | | |

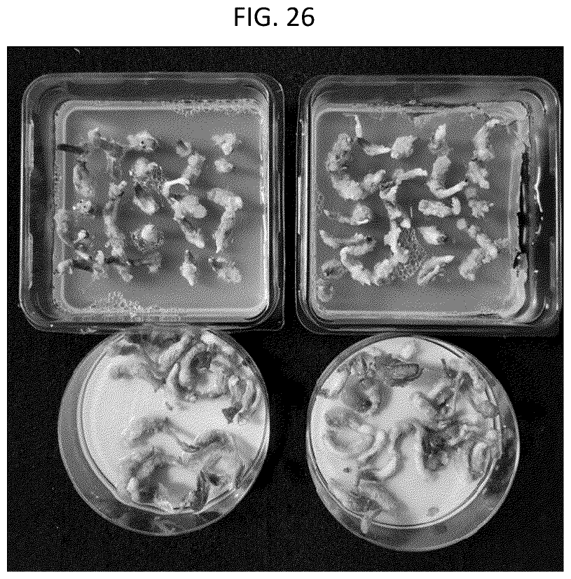

FIG. 26 shows okra meristem explants on 25 ppm spec (left); 50 ppm spec (right) on solid (top) or liquid (bottom) Hemp node media (˜3 weeks post inoculation). Okra regeneration appears enhanced on liquid media relative to solid media, as in Cannabis.

### Example 6

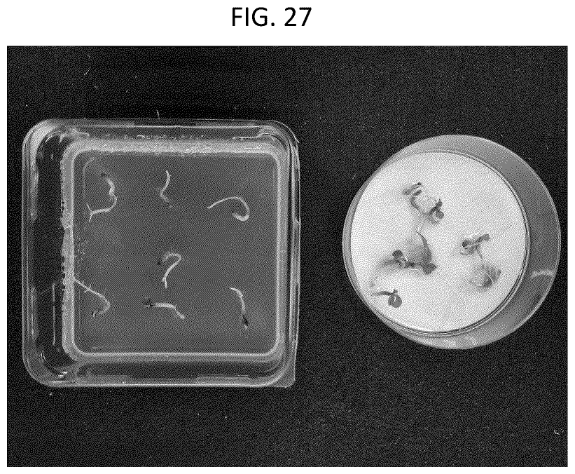

The following example describes efficient transformation in Cotton (Gossypium hirsutum, L.). We also examined phenotypes of Cotton meristem explants on non-selective solid B5 (right), and on liquid B5 (left) after ˜2 weeks (FIG. 27) and noted a greater rate of regeneration/greening in the Cotton explants on liquid media, suggesting possible utility in this crop as well.

### Example 7

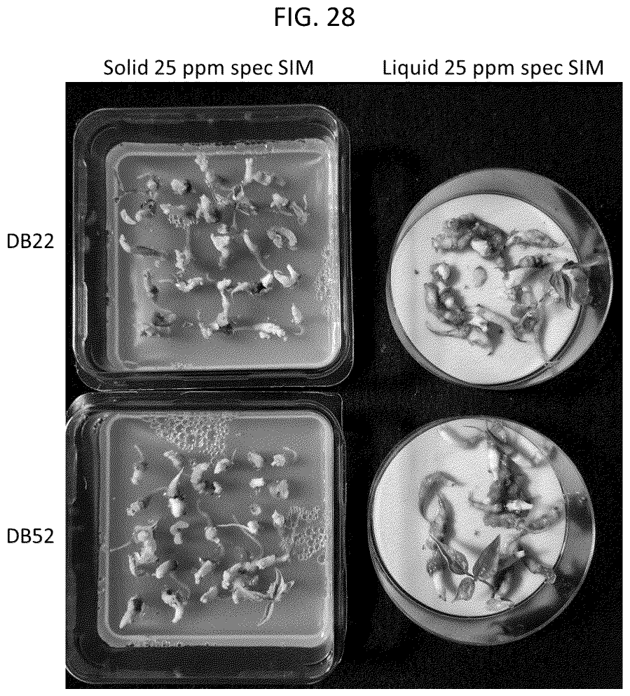

The following example describes efficient transformation in Cowpea (Vigna unguiculata, L.). We used a variant of the Cowpea transformation process described by Che et. al. to examine the possibility of using a liquid selection media for Cowpea meristem transformation. The Liquid Cowpea shoot induction media (SIM) is analogous to the Efficient Cannabis meristem method, but differs from the protocol described in Table 7 by replacing meta-topolin with 0.5 mg/L BAP and 0.5 mg/L kinetin. The liquid Cowpea SIM differs from Che's SIM by removing agar, has no MES, and has pH 5.7 rather than Che's 5.6. We were able to establish proof of concept of generating TO Cowpea seedlingswith DB22 and DB52. and a hydroponic/liquid selection media regime analogous to Efficient Cannabis meristem method. However, the liquid selection media (right) did not appear advantageous relative to standard semisolid selection media regime (left) without additional changes, as shown in FIG. 28 and Table 16.

TABLE 16
Pilot Tests of Liquid Media in Cowpea

| | | | | | | | | | # | | | |
|---|---|---|---|---|---|---|---|---|---|---|---|---|
| | | | | | | | | | greening | | | |
| | | | | | | | | | explants | | | |
| | | | | | | Explants | First | | transferred | | | |
| | | | | | | to 25 ppm | Selection | | to WPM | | # | |
| | Genotype/ | | | | Co- | spec | Media (24 h | Decap- | 27 C. 16/8 | # | rooting | putative |
| Species | Line | Strain | Binary | Experiment | culture | SIM cct | light 26 C.) | itated | photoperiod | shoots | shoots | TF |
| Cowpea | IT86D- | Ar18r12v | DB22 | Cowpea | 2 d cc in | 125 | 25 ppm | Jun. 2, | 22 | 22 | 15 | 12% |
| | 1010 | | | 5/24-1 | 23 C. 16/8 | | spec | 2023 | | | | |
| | | | | | photoperiod | | SIM cct | | | | | |
| | | | | | (Cowpea | | | | | | | |
| | | | | | INF media, | | | | | | | |
| | | | | | 700 uL/ | | | | | | | |
| | | | | | plate) | | | | | | | |
| Cowpea | IT86D- | Ar18r12v | DB52 | Cowpea | 2 d cc in | 125 | 25 ppm | Jun. 2, | 9 | 7 | 5 | 4% |
| | 1010 | | | 5/24-2 | 23 C. 16/8 | | spec | 2023 | | | | |
| | | | | | photoperiod | | SIM cct | | | | | |
| | | | | | (Cowpea | | | | | | | |
| | | | | | INF media, | | | | | | | |
| | | | | | 700 uL/ | | | | | | | |
| | | | | | plate) | | | | | | | |
| Cowpea | IT86D- | Ar18r12v | DB22 | Cowpea | 2 d cc in | 110 | LIQUID | Jun. 2, | 6 | 5 | 5 | 5% |
| | 1010 | | | 5/24-3 | 23 C. 16/8 | | 25 ppm | 2023 | | | | |
| | | | | | photoperiod | | spec cct | | | | | |
| | | | | | (Cowpea | | | | | | | |
| | | | | | INF media, | | | | | | | |
| | | | | | 700 uL/ | | | | | | | |
| | | | | | plate) | | | | | | | |
| Cowpea | IT86D- | Ar18r12v | DB52 | Cowpea | 2 d cc in | 110 | LIQUID | Jun. 2, | 2 | 2 | 0 | 0% |
| | 1010 | | | 5/24-4 | 23 C. 16/8 | | 25 ppm | 2023 | | | | |
| | | | | | photoperiod | | spec cct | | | | | |
| | | | | | (Cowpea | | | | | | | |
| | | | | | INF media, | | | | | | | |
| | | | | | 700 uL/ | | | | | | | |
| | | | | | plate) | | | | | | | |

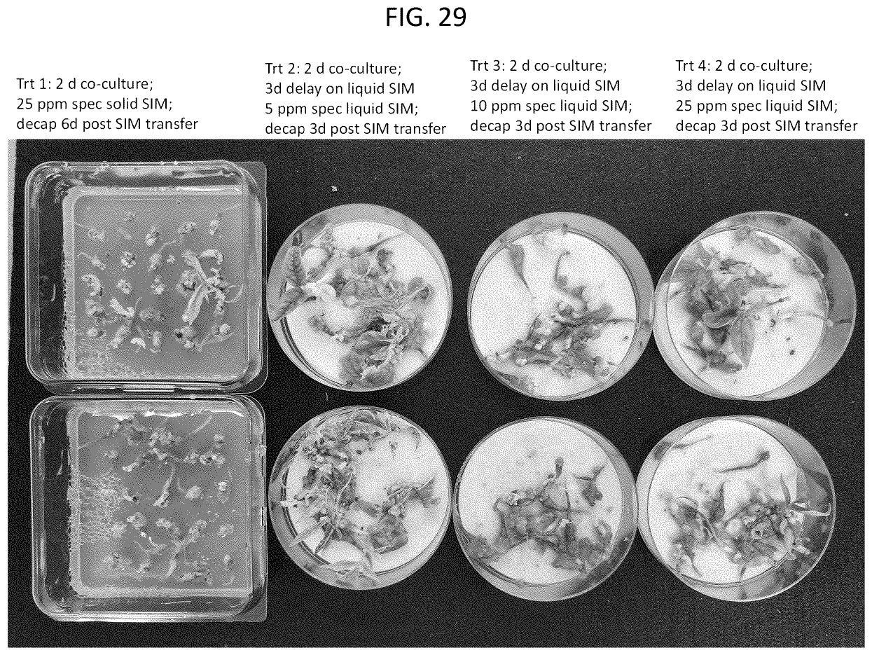

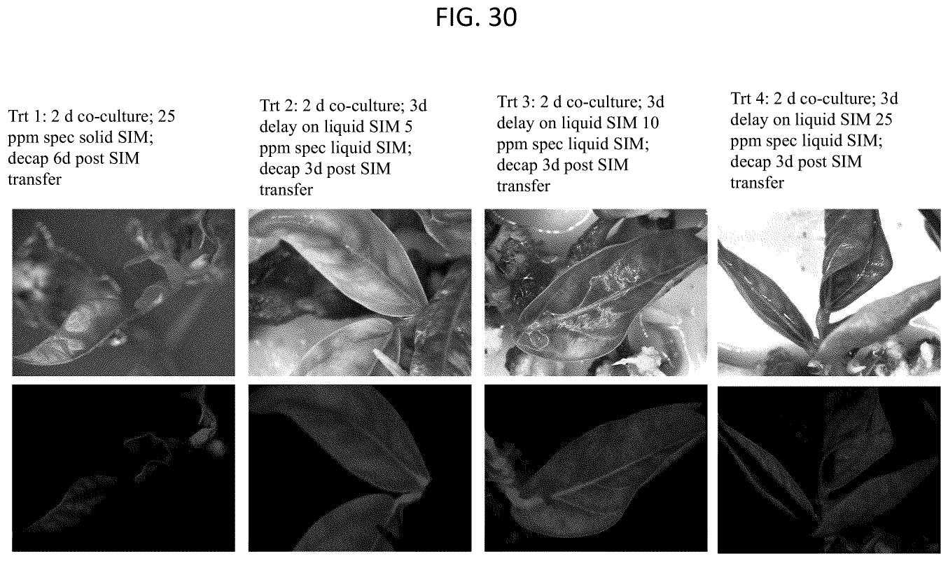

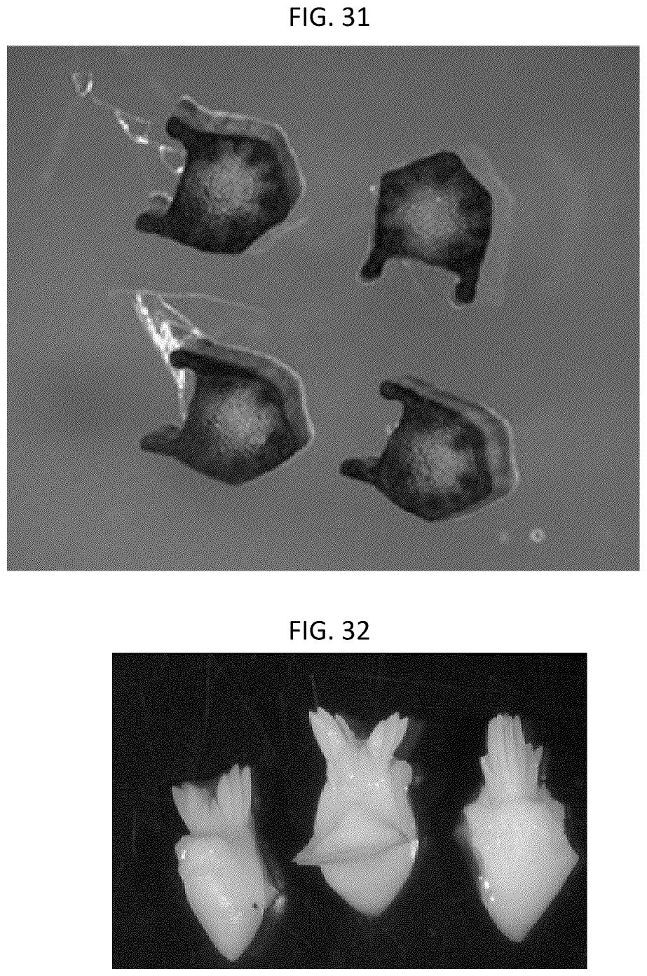

We then tested Cowpea variety “Crowder Pea” with a brief liquid delay phase. For example, a 3d delay means 3 days on liquid media without selection (after co-culture), followed by varying levels of selection on liquid media. There appeared to be some advantage in using a 3 day liquid delay phase followed by liquid selection at 25 mg/L spectinomycin (FIG. 29, Table 17). Stable tdTomato expression in Crowder Pea events generated on solid media and on liquid media using delay phase is shown in FIG. 30. We examined presence/absence of GUS expression in the vascular bundles of cross-sectioned cowpea petioles to predict germline status (FIG. 31). Explants from these experiments were all inoculated with Agrobacterium rhizogenes strain 18r12v (Ar18r12v)/DICOTBINARY22.

TABLE 17
Transformation metrics in Cowpea with Liquid Media and Delay Phase

| | | Explants | | | # greening | | | | | |
|---|---|---|---|---|---|---|---|---|---|---|
| | | to 25 ppm | | | explants | Shoots | | | #TO0 | |
| | | spec SIM, | First | Second | transferred | harvested to | | | plants | Predicted |
| | Geno | 26 C. 24 h | Selection | Selection | to WPM | 25-50 ppm | | | predicted | germline |
| | type/ | light after | Media (24 h | Media (24 h | 27 C. 16/8 | spec WPM | #rooting | Putative | germline | TF |
| Species | Line | co-culture | light 26 C.) | light 26 C.) | photoperiod | rooting | shoots | TF | (petiole) | (petiole) |
| Cowpea | Crowder | 100 | 25 ppm | n/a | 8 | 4 | 2 | 2.0% | 1 | 1.0% |
| | Pea | | spec SIM | | | | | | | |
| | | | cct | | | | | | | |
| | | | (embedded) | | | | | | | |
| Cowpea | Crowder | 90 | 3 d | 10 ml of 5 | 20 | 16 | 3 | 3.3% | 1 | 1.1% |
| | Pea | | delay: | ppm spec | | | | | | |
| | | | 0 ppm | LIQUID SIM | | | | | | |
| | | | LIQUID | cct | | | | | | |
| | | | spec | | | | | | | |
| | | | SIM cct | | | | | | | |
| | | | (5 ml) | | | | | | | |
| Cowpea | Crowder | 90 | 3 d | 10 ml of 10 | 6 | 6 | 3 | 3.3% | 1 | 1.1% |
| | Pea | | delay: | ppm spec | | | | | | |
| | | | 0 ppm | LIQUID SIM | | | | | | |
| | | | LIQUID | cct | | | | | | |
| | | | spec | | | | | | | |
| | | | SIM cct | | | | | | | |
| | | | (5 ml) | | | | | | | |
| Cowpea | Crowder | 90 | 3 d | 10 ml of 25 | 8 | 6 | 4 | 4.4% | 4 | 4.4% |
| | Pea | | delay: | ppm spec | | | | | | |
| | | | 0 ppm | LIQUID SIM | | | | | | |
| | | | LIQUID | cct | | | | | | |
| | | | spec | | | | | | | |
| | | | SIM cct | | | | | | | |
| | | | (5 ml) | | | | | | | |

### Example 8

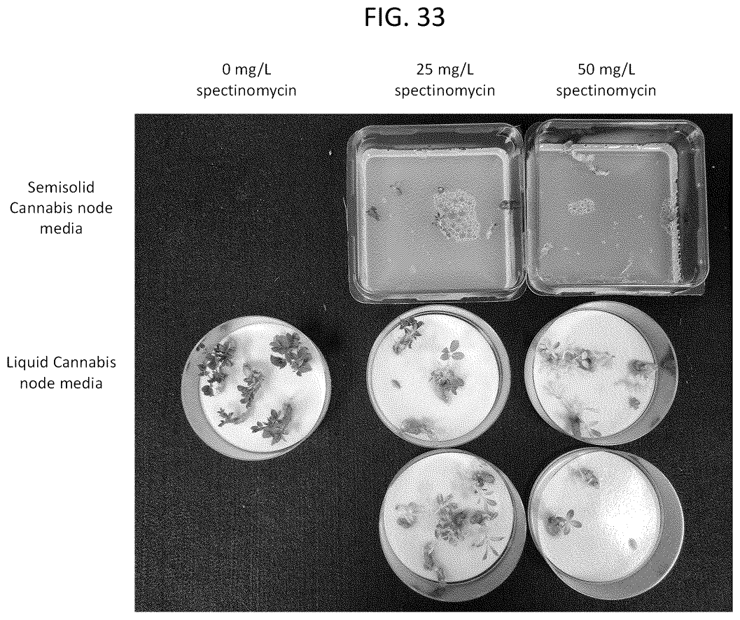

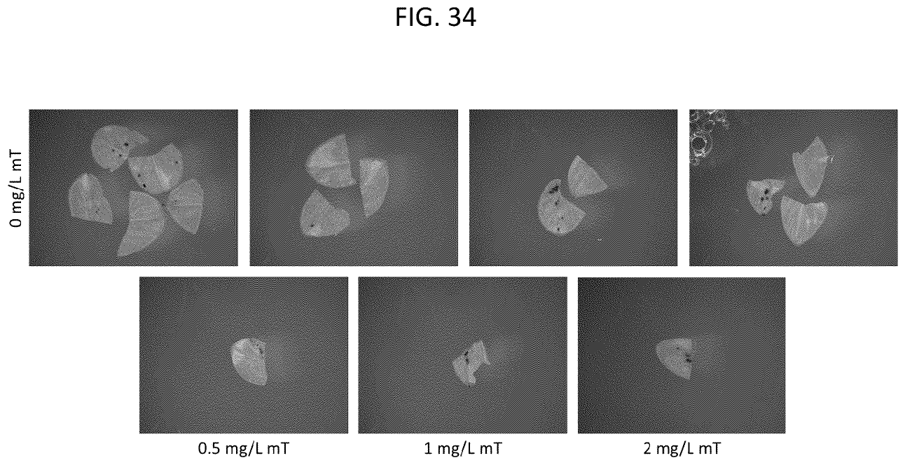

The following example describes efficient transformation in Peanut (Arachis hypogaea, L.). We also ran small experiments of Peanut meristem explants (FIG. 32) on solid and liquid MS-based Cannabis node selection medias post co-culture with three rates of spectinomycin (0 mg/L, 25 mg/L and 50 mg/L) and noted a greater rate of regeneration in the Peanut meristem explants on liquid selection media compared to explants on solid selection media approximately 2.5 weeks after inoculation (FIG. 33). We did not recover stable TO Peanut plants from these pilot tests, but did recover regenerating highly chimeric Peanut plants stably expressing GUS (FIG. 34), suggesting feasibility of this strategy to those skilled in the art.

What is claimed:

1. A method of transforming an explant with an exogenous nucleic acid, the method comprising:
a) excising the explant from a seed by removing the seed coat and optionally cotyledons,
b) introducing the exogenous nucleic acid into the explant, and
c) culturing the explant on a liquid selection medium to select for a transformed explant.

2. The method of claim 1, wherein the seed is selected from the group consisting of a Cannabis seed, an Abelmoschus seed, a Gossypium seed, a Vigna seed, and an Arachis seed.

3. The method of claim 1, further comprising sanitizing the seed prior to step (a).

4. The method of claim 1, further comprising hydrating the seed in a hydration medium prior to step (a).

5. The method of claim 1, wherein the explant comprises both primary leaves.

6. The method of claim 1, wherein the exogenous nucleic acid is introduced by co-culturing the explant in a co-culture medium with Agrobacterium comprising the exogenous nucleic acid for about 1 to 6 days.

7. The method of claim 6, wherein the co-culture medium comprises one or more growth regulators.

8. The method of claim 6, wherein the co-culture medium is the medium described in Table 6.

9. The method of claim 1, further comprising force treating the explant prior to or following step (b).

10. The method of claim 9, wherein the force treatment is selected from the group consisting of sonication, vortexing, centrifugation, heat-shock, pressure, vacuum infiltration, and addition of chemicals.

11. The method of claim 1, wherein the liquid selection medium comprises 10-150 mg/L spectinomycin.

12. The method of claim 11, wherein the liquid selection medium is selected from the group consisting of the medium described in Table 7, B5 medium, DKW medium, WPM-based medium, MS salts-based medium, and ½×MS salts-based medium.

13. The method of claim 1, wherein callus formation is minimized compared to callus formation using a solid selection medium.

14. The method of claim 1, wherein the method further comprises:
d) culturing the transformed explant on a rooting medium.

15. The method of claim 14, wherein the rooting medium comprises 5-100 mg/L spectinomycin.

16. The method of claim 1, wherein the method produces greenhouse-ready plantlets in fewer than 100 days after introducing the exogenous nucleic acid into the explant.

17. The method of claim 1, wherein the method results in a transformation frequency of greater than 1%.

18. The method of claim 1, wherein the exogenous nucleic acid encodes or includes a guide RNA.

19. A transformed explant produced by the method of claim 1.

20. A plant grown from the explant of claim 19, wherein the plant is a T1 plant.

21. A seed produced from the plant of claim 20.
